(19) United States
(12) Patent Application Publication
(10) Pub. No.: US 2023/0227580 A1
(43) Pub. Date: Jul. 20, 2023

(54) ANTI-VWF ANTIBODIES AND USES THEREOF

(71) Applicants: MONASH UNIVERSITY, Clayton, Victoria (AU); BAKER HEART AND DIABETES INSTITUTE, Melbourne, Victoria (AU)

(72) Inventors: Christoph HAGEMEYER, Clayton, Victoria (AU); Erik WESTEIN, Clayton, Victoria (AU); Thomas HOEFER, Clayton, Victoria (AU); Robert ANDREWS, Clayton, Victoria (AU); Elizabeth GARDINER, Clayton, Victoria (AU)

(73) Assignees: MONASH UNIVERSITY, Clayton, Victoria (AU); BAKER HEART AND DIABETES INSTITUTE, Melbourne, Victoria (AU)

(21) Appl. No.: 18/000,557

(22) PCT Filed: Jun. 25, 2021
(86) PCT No.: PCT/AU2021/050676

§ 371 Date: Dec. 2, 2022

(30) Foreign Application Priority Data
Jun. 26, 2020 (AU) 2020902148

(51) Int. Cl. C07K 16/36; A61P 7/02

(52) U.S. Cl. CPC C07K 16/36; A61P 7/02; C07K 2317/76; C07K 2317/565; C07K 2317/567; C07K 2317/622

(57) ABSTRACT

The invention also relates to antigen binding proteins and related fragments thereof for binding Von Willebrand factor (VWF) and uses thereof. In one aspect, the present invention provides an antigen binding protein comprising an antigen binding domain that binds to or specifically binds to Von Willebrand factor (VWF) under shear gradient conditions. Preferably, the antigen binding protein comprises an antigen binding domain that does not bind to VWF under constant shear conditions.

### FIELD OF THE INVENTION

The invention relates to antigen binding proteins and related fragments thereof for binding Von Willebrand factor (VWF) and uses thereof.

### RELATED APPLICATION

This application claims priority from Australian provisional application AU 2020902148, the entire contents of which are hereby incorporated by reference.

### BACKGROUND OF THE INVENTION

Atherothrombotic events precipitating myocardial infarction and stroke are the most common causes of death worldwide. These events are triggered by intraluminal plaque rupture causing an inappropriate platelet response which leads to occlusion of the vessel lumen. As a preventative measure, prescribed antiplatelet drugs such as aspirin and clopidogrel target major platelet activation pathways such as the TXA₂- and ADP-P2Y₁₂-pathways, thereby reducing platelet reactivity. While this is a clinically proven strategy to reduce cardiovascular events, this approach can also cause serious side effects such as bleeding, as the targeted pathways also play a critical role in normal haemostasis thereby partly offsetting the benefits gained from these drugs. Thus, a strategy to uncouple thrombosis from haemostasis to target one without the other is an urgent unmet clinical need.

Recent insights into the haemodynamic regulation of thrombus formation has led to the concept that a key difference between thrombosis and haemostasis is the rapidly changing shear conditions at the site of a cardiovascular insult. Platelet aggregates formed during a haemostatic response typically do not grow beyond 50% of the vessel lumen, a restriction that is insufficient to markedly change the shear rates at the site of injury. Plaque rupture with its release of highly thrombogenic content may cause formation of a large thrombus and severely constricts of the vessel lumen. This in turn causes local steep increases in blood shear rates, also termed shear rate gradients (SRG) or shear stress gradients (SSG).

There is therefore a need for new or improved approaches for the treatment or prevention of conditions associated with pathological thrombus formation.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides an antigen binding protein comprising an antigen binding domain that binds to or specifically binds to Von Willebrand factor (VWF) under shear gradient conditions. Preferably, the antigen binding protein comprises an antigen binding domain that does not bind to VWF under constant shear conditions.

Preferably, the antigen binding protein of the invention binds to or specifically binds to human VWF, preferably the A1 domain. Preferably, the antigen binding protein binds to or specifically binds to a human VWF molecule comprising, consisting essentially of or consisting of an amino acid sequence as shown in SEQ ID NO: 47.

Preferably, an antigen binding protein of the invention inhibits or reduces platelet - VWF interaction or platelet deposition.

Preferably, an antigen binding protein of the invention inhibits or reduces thrombus formation.

Determining whether an antigen binding protein reduced or inhibits thrombus formation or platelet deposition at shear gradient conditions but not constant shear conditions may be determined as outlined in the Examples, particularly Example 4. In any aspect, an antigen binding protein of the invention may inhibit thrombus formation and/or platelet deposition at shear rate gradients of 250-2,000 s⁻¹ or 187-1,500 s⁻¹ (or any other shear rate gradients described herein, including the Examples) but not at constant shear conditions of 1,500 s⁻¹ or 2,000 s⁻¹ (or any other constant shear conditions described herein, including the Examples).

In one embodiment, an antigen binding protein of the invention may inhibit thrombus formation and/or platelet deposition to a significantly greater extent than the platelet GPlbα inhibitor OS-1. Preferably, as determined by an assay as described herein, including the Examples, particularly Example 4.

The invention provides an antigen binding protein that binds to or specifically binds to VWF and wherein the antigen binding protein competitively inhibits binding of the A1 antibody (i.e., comprising a VH comprising a sequence set forth in SEQ ID NO: 11 and a VL comprising a sequence set forth in SEQ ID NO: 10) to VWF.

The invention also provides an antigen binding protein that binds to the same epitope on VWF as an antibody that comprises a VH domain comprising the amino acid sequence as set forth in SEQ ID NO: 11.

The invention also provides an antigen binding protein that binds to the same epitope on VWF as an antibody that comprises a VH domain comprising the amino acid sequence as set forth in SEQ ID NO: 11 and a VL domain comprising the amino acid sequence as set forth in SEQ ID NO: 10.

The invention provides an antigen binding protein for binding to VWF, the antigen binding protein comprising:
FR1 - CDR1 - FR2 - CDR2 - FR3 - CDR3 - FR4, and
FR1a - CDR1a - FR2a - CDR2a - FR3a - CDR3a - FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the framework regions or complementarity determining regions are as described herein.

The invention provides an antigen binding protein for binding to VWF, the antigen binding protein comprising:
FR1 - CDR1 - FR2 - CDR2 - FR3 - CDR3 - FR4, and
FR1a - CDR1a - FR2a - CDR2a - FR3a - CDR3a - FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

In any embodiment, the antigen binding protein comprises a linker region such that the protein comprises:

FR1 - CDR1 - FR2 - CDR2 - FR3 - CDR3 - FR4 - linker - FR1a- CDR1a - FR2a - CDR2a - FR3a - CDR3a - FR4a wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

As defined herein, the linker may be a chemical, one or more amino acids (including a polypeptide), or a disulphide bond formed between two cysteine residues.

The invention provides an antigen binding protein comprising, consisting essentially of or consisting of an amino acids sequence of (in order of N to C terminus or C to N terminus) SEQ ID NOs: 10 and 11.

The present invention also provides an antigen binding protein comprising an antigen binding domain of an antibody, wherein the antigen binding domain binds to or specifically binds to VWF, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4, a CDR2 comprising a sequence at least about 80%, at least 85%, at least 90%, at least 92%, at least 95%, at least 97%, at least 99% identical to a sequence set forth in SEQ ID NO:5 and a CDR3 comprising a sequence at least about 80%, at least 85%, at least 90%, at least 92%, at least 95%, at least 97%, at least 99% identical to a sequence set forth in SEQ ID NO: 6;
(ii) a VH comprising a sequence at least about 95% or 96% or 97% or 98% or 99% identical to a sequence set forth in SEQ ID NO: 11;
(iii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(iv) a VL comprising a sequence at least about 95% or 96% or 97% or 98% or 99% identical to a sequence set forth in SEQ ID NO: 10;
(v) a VH comprising a CDR1 comprising a sequence set forth in SEQ ID NO: 4, a CDR2 comprising a sequence set forth in SEQ ID NO: 5 and a CDR3 comprising a sequence set forth in SEQ ID NO: 6;
(vi) a VH comprising a sequence set forth in SEQ ID NO: 11;
(vii) a VL comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3;
(viii) a VL comprising a sequence set forth in SEQ ID NO: 10;
(ix) a VH comprising a CDR1 comprising a sequence set forth in SEQ ID NO: 4, a CDR2 comprising a sequence set forth in SEQ ID NO: 5 and a CDR3 comprising a sequence set forth in SEQ ID NO: 6; and a VL comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3; or
(x) a VH comprising a sequence set forth in SEQ ID NO: 11 and a VL comprising a sequence set forth in SEQ ID NO: 10.

In one embodiment, the antigen binding domain comprises (i) and (iii).

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:27, a FR2 comprising a sequence at least about 80%, at least 85%, at least 90%, at least 92%, at least 95%, at least 97%, at least 99% identical to a sequence set forth in SEQ ID NO:28, a FR3 comprising a sequence at least about 80%, at least 85%, at least 90%, at least 92%, at least 95%, at least 97%, at least 99% identical to a sequence set forth in SEQ ID NO: 29, and a FR4 comprising a sequence at least about 80%, at least 85%, at least 90%, at least 92%, at least 95%, at least 97%, at least 99% identical to a sequence set forth in SEQ ID NO: 30;
(ii) a VL comprising a FR1 comprising a sequence at least about 80%, at least 85%, at least 90%, at least 92%, at least 95%, at least 97%, at least 99% identical to a sequence set forth in SEQ ID NO: 23, a FR2 comprising a sequence at least about 80%, at least 85%, at least 90%, at least 92%, at least 95%, at least 97%, at least 99% identical to a sequence set forth in SEQ ID NO: 24, a FR3 comprising a sequence at least about 80%, at least 85%, at least 90%, at least 92%, at least 95%, at least 97%, at least 99% identical to a sequence set forth in SEQ ID NO: 25, and a FR4 comprising a sequence at least about 80%, at least 85%, at least 90%, at least 92%, at least 95%, at least 97%, at least 99% identical to a sequence set forth in SEQ ID NO: 26;
(iii) a VH comprising a FR1 comprising a sequence set forth in SEQ ID NO: 27, a FR2 comprising a sequence set forth in SEQ ID NO: 28, a FR3 comprising a sequence set forth in SEQ ID NO: 29, and a FR4 comprising a sequence set forth in SEQ ID NO: 30;
(iv) a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26; or
(v) a VH comprising a FR1 comprising a sequence set forth in SEQ ID NO: 27, a FR2 comprising a sequence set forth in SEQ ID NO: 28, a FR3 comprising a sequence set forth in SEQ ID NO: 29, and a FR4 comprising a sequence set forth in SEQ ID NO: 30; and a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26.

The present invention also provides an antigen binding protein comprising an antigen binding domain of an antibody, wherein the antigen binding domain binds to or specifically binds to VWF, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:7, a CDR2 comprising a sequence at least about 80%, at least 85%, at least 90%, at least 92%, at least 95%, at least 97%, at least 99% identical to a sequence set in SEQ ID NO:8 and a CDR3 comprising a sequence at least about 80%, at least 85%, at least 90%, at least 92%, at least 95%, at least 97%, at least 99% identical to a sequence set forth in SEQ ID NO: 9;
(ii) a VH comprising a sequence at least about 95% or 96% or 97% or 98% or 99% identical to a sequence set forth in SEQ ID NO: 11;
(iii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(iv) a VL comprising a sequence at least about 95% or 96% or 97% or 98% or 99% identical to a sequence set forth in SEQ ID NO: 10;
(v) a VH comprising a CDR1 comprising a sequence set forth in SEQ ID NO: 7, a CDR2 comprising a sequence set forth in SEQ ID NO: 8 and a CDR3 comprising a sequence set forth in SEQ ID NO: 9;
(vi) a VH comprising a sequence set forth in SEQ ID NO: 11;
(vii) a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3;
(viii) a VL comprising a sequence set forth in SEQ ID NO: 10;
(ix) a VH comprising a CDR1 comprising a sequence set forth in SEQ ID NO: 7, a CDR2 comprising a sequence set forth in SEQ ID NO: 8 and a CDR3 comprising a sequence set forth in SEQ ID NO: 9; and a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3; or
(x) a VH comprising a sequence set forth in SEQ ID NO: 11 and a VL comprising a sequence set forth in SEQ ID NO: 10.

In one embodiment, the antigen binding domain comprises (i) and (iii).

In any aspect of the invention, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:31, a FR2 comprising a sequence at least about 80%, at least 85%, at least 90%, at least 92%, at least 95%, at least 97%, at least 99% identical to a sequence set forth in SEQ ID NO:32, a FR3 comprising a sequence at least about 80%, at least 85%, at least 90%, at least 92%, at least 95%, at least 97%, at least 99% identical to a sequence set forth in SEQ ID NO: 33, and a FR4 comprising a sequence at least about 80%, at least 85%, at least 90%, at least 92%, at least 95%, at least 97%, at least 99% identical to a sequence set forth in SEQ ID NO: 34;
(ii) a VL comprising a FR1 comprising a sequence at least about 80%, at least 85%, at least 90%, at least 92%, at least 95%, at least 97%, at least 99% identical to a sequence set forth in SEQ ID NO: 23, a FR2 comprising a sequence at least about 80%, at least 85%, at least 90%, at least 92%, at least 95%, at least 97%, at least 99% identical to a sequence set forth in SEQ ID NO: 24, a FR3 comprising a sequence at least about 80%, at least 85%, at least 90%, at least 92%, at least 95%, at least 97%, at least 99% identical to a sequence set forth in SEQ ID NO: 25, and a FR4 comprising a sequence at least about 80%, at least 85%, at least 90%, at least 92%, at least 95%, at least 97%, at least 99% identical to a sequence set forth in SEQ ID NO: 26;
(iii) a VH comprising a FR1 comprising a sequence set forth in SEQ ID NO: 31, a FR2 comprising a sequence set forth in SEQ ID NO: 32, a FR3 comprising a sequence set forth in SEQ ID NO: 33, and a FR4 comprising a sequence set forth in SEQ ID NO: 34;
(iv) a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26; or
(v) a VH comprising a FR1 comprising a sequence set forth in SEQ ID NO: 31, a FR2 comprising a sequence set forth in SEQ ID NO: 32, a FR3 comprising a sequence set forth in SEQ ID NO: 33, and a FR4 comprising a sequence set forth in SEQ ID NO: 34; and a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26.

The present invention also provides an antigen binding protein comprising an antigen binding domain of an antibody, wherein the antigen binding domain binds to or specifically binds to VWF, wherein the antigen binding domain comprises a CDR1, CDR2 and CDR3 from the light chain variable region shown in SEQ ID NO: 10 and a CDR1, CDR2 and CDR3 from the heavy chain variable region shown in SEQ ID NO: 11.

As described herein, the antigen binding protein may be in the form of:
(i) a single domain antibody (sdAb);
(ii) a single chain Fv fragment (scFv);
(iii) a dimeric scFv (di-scFv);
(iv) one of (i) to (iii) linked to a constant region of an antibody, Fc or a heavy chain constant domain (CH) 2 and/or CH3; or
(v) one of (i) to (iv) linked to a protein that binds to an immune effector cell.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3; or
(ix) one of (i) to (viii) linked to a protein that binds to an immune effector cell.

The foregoing antigen binding proteins can also be referred to as antigen binding domains of antibodies.

Preferably, an antigen binding protein as described herein is an antibody or antigen binding fragment thereof. Typically, the antigen binding protein is an antibody, for example, a monoclonal antibody.

The present invention also provides an VWF binding antibody comprising a light chain variable region and a heavy chain variable region,
wherein said light chain variable region comprises:
a CDR L1 as set forth in SEQ ID NO: 1, a CDR L2 as set forth in SEQ ID NO: 2 and a CDR L3 as set forth in SEQ ID NO: 3; and
wherein said heavy chain variable region comprises:
a CDR H1 as set forth in SEQ ID NO: 4, a CDR H2 as set forth in SEQ ID NO:5, and a CDR H3 as set forth in SEQ ID NO: 6.

The present invention also provides an VWF binding antibody comprising a light chain variable region and a heavy chain variable region,
wherein said light chain variable region comprises:
a CDR L1 as set forth in SEQ ID NO: 1, a CDR L2 as set forth in SEQ ID NO: 2 and a CDR L3 as set forth in SEQ ID NO: 3; and
wherein said heavy chain variable region comprises:
a CDR H1 as set forth in SEQ ID NO: 7, a CDR H2 as set forth in SEQ ID NO: 8, and a CDR H3 as set forth in SEQ ID NO: 9.

In any aspect of the invention, a VWF antibody comprises a light chain variable region that comprises the sequence of SEQ ID NO: 10.

In any aspect of the invention, a VWF antibody comprises a heavy chain variable region that comprises the sequence of SEQ ID NO: 11.

In any aspect of the invention, a VWF antibody comprises a light chain variable region that comprises a FR L1 as set forth in SEQ ID NO: 23, FR L2 as set forth in SEQ ID NO: 24, a FR L3 as set forth in SEQ ID NO: 25 and a FR L4 as set forth in SEQ ID NO: 26.

In any aspect of the invention, a VWF antibody comprises a heavy chain variable region that comprises a FR H1 as set forth in SEQ ID NO: 27, FR H2 as set forth in SEQ ID NO: 28, a FR H3 as set forth in SEQ ID NO: 29 and a FR H4 as set forth in SEQ ID NO: 30.

In any aspect of the invention, a VWF antibody comprises a heavy chain variable region that comprises a FR H1 as set forth in SEQ ID NO: 31, FR H2 as set forth in SEQ ID NO: 32, a FR H3 as set forth in SEQ ID NO: 33 and a FR H4 as set forth in SEQ ID NO: 34.

As used herein, the complementarity determining region sequences (CDRs) of an antigen binding protein of the invention are defined according to the Kabat or Chothia numbering systems.

Reference herein to a protein or antibody that “binds to” VWF provides literal support for a protein or antibody that “binds specifically to” or “specifically binds to” VWF.

The present invention also provides antigen binding domains or antigen binding fragments of the foregoing antibodies.

The invention also provides a fusion protein comprising an antigen binding protein, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

In another aspect, the present invention provides a method of reducing pathological thrombus formation, the method comprising administering an antigen binding protein of the invention as described herein to an individual in need thereof, thereby reducing pathological thrombus formation.

In another aspect, the present invention provides a method for inhibiting thrombosis without compromising haemostasis in a subject in need thereof, the method comprising administering an antigen binding protein of the invention as described herein, thereby inhibiting thrombosis without compromising haemostasis in the subject.

In another aspect, the present invention provides a method for inhibiting a pathological thrombotic condition in a subject in need thereof, the method comprising administering an antigen binding protein of the invention as described herein, thereby inhibiting a pathological thrombotic condition in the subject. Preferably, the pathological thrombotic condition is any one described herein.

As used herein a pathological thrombotic condition may be one associated with, or caused by, the formation or presence of a thrombus or blood clot in a blood vessel.

The antigen binding proteins of the invention find particular application in treating pathological thrombotic conditions in a subject, wherein an effect on haemostasis should or needs to be avoided.

The present invention also provides a method for inhibiting thrombosis in a subject in need thereof comprising administering to the subject an effective dose of an antigen binding protein of the invention described herein, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.

In another aspect, the present invention provides use of an antigen binding protein of the invention as described herein in the manufacture of a medicament for reducing pathological thrombus formation in a subject in need thereof.

In another aspect, the present invention provides use of an antigen binding protein of the invention as described herein in the manufacture of a medicament for inhibiting thrombosis without compromising haemostasis in a subject in need thereof.

In another aspect, the present invention provides use of an antigen binding protein of the invention as described herein in the manufacture of a medicament for inhibiting a pathological thrombotic condition in a subject in need thereof. Preferably, the pathological thrombotic condition is any one described herein.

In another aspect, the present invention provides use of antigen binding protein of the invention described herein in the manufacture of a medicament for inhibiting thrombosis in a subject in need thereof, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.

In another aspect, the present invention provides an antigen binding protein of the invention as described herein for use in reducing pathological thrombus formation in a subject in need thereof.

In another aspect, the present invention provides an antigen binding protein of the invention as described herein for use in inhibiting thrombosis without compromising haemostasis in a subject in need thereof.

In another aspect, the present invention provides an antigen binding protein of the invention as described herein for use in inhibiting a pathological thrombotic condition in a subject in need thereof. Preferably, the pathological thrombotic condition is any one described herein.

In another aspect, the present invention provides an antigen binding protein of the invention described herein for use in inhibiting thrombosis in a subject in need thereof, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.

The invention also provides an antibody for binding to an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein.

The invention also provides a nucleic acid encoding an antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding proteins, the expression construct may comprise a promoter linked to a nucleic acid encoding that polypeptide chain.

In examples directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,

wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,

wherein the first and second polypeptides associate to form an antigen binding protein of the present invention.

Examples of cells of the present invention include bacterial cells, yeast cells, insect cells or mammalian cells.

The invention also provides a pharmaceutical composition comprising an antigen binding protein, or comprising a CDR and/or FR sequence as described herein, or an immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody, fusion protein, or conjugate as described herein and a pharmaceutically acceptable carrier, diluent or excipient.

The invention also provides a diagnostic composition comprising an antigen binding protein, or comprising a CDR and/or FR sequence as described herein, or antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein, a diluent and optionally a label.

The invention also provides a kit or article of manufacture comprising an antigen binding protein, or comprising a CDR and/or FR sequence as described herein or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

An antigen binding protein or antibody as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example a protein or antibody as described herein or a composition of a protein or antibody as described herein, comprises a heavy chain constant region, comprising a stabilized heavy chain constant region, comprising a mixture of sequences fully or partially with or without the C-terminal lysine residue.

In one example, an antibody of the invention comprises a VH disclosed herein linked or fused to an IgG4 constant region or stabilized IgG4 constant region (e.g., as discussed above) and the VL is linked to or fused to a kappa light chain constant region.

The functional characteristics of an antigen binding protein of the invention will be taken to apply mutatis mutandis to an antibody of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

The invention also provides a cell comprising a vector or nucleic acid molecule described herein.

The invention also provides an animal or tissue derived therefrom comprising a cell described herein.

The present invention also provides a kit for use or when used in any method of the invention described herein.

Preferably the kit comprises an antigen binding protein as described herein, optionally with instructions for use in a method of the invention.

The antigen binding protein contained in the kit may include a detectable label. Alternatively, or in addition, the antigen binding protein may be bound to a solid support.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. SRGs increase VWF-GPIb bond strength and reduce platelet-rolling velocity. A. Complex flow dynamics were studied in microfluidics exhibiting a semi-circular stenotic site with a diameter of 600 µm which reduced the channel width from 300 µm to 60 µm. B. Computational fluid dynamics identified the stenosis apex as a zone with an eight-fold increase in shear compared to the inlet, followed by a negative but equal drop in shear rate in the stenosis outlet. C. Abciximab-treated (20 µg ml⁻¹), citrated whole blood, labelled with 0.5 µg ml⁻¹ DiOC₆ was perfused over a collagen III-coated surface to promote binding of blood borne VWF under flow and subsequent platelet adhesion. Input shear rate was set to 300 s⁻¹ which then increased to 2,400 s⁻¹ in the apex of the stenosis. Control channels with straight walls were used to generate constant shear rate of 2,400 s⁻¹. D. Platelet rolling velocity on VWF-collagen III was assessed at SRGs (platelets rolling through the apex of the stenotic site) or constant shear (upstream of stenotic site) using particle tracking software. Platelet rolling velocities were divided into three bins (0-3 µm s⁻¹; >3-6 µm s⁻¹; >6 µm s⁻¹) and presented as relative frequencies. E. Surface coverage of abciximab-treated, citrated whole blood, labelled with DiOC₆ perfused over a collagen III-coated surface. Input shear rates ranged from 125 s⁻¹ up to 8,000 s⁻¹ for stenotic channels, resulting in peak shear rates of 1,000 s⁻¹ to 36,000 s⁻¹ in the apex of the stenosis; or matched shear rates in a parallel control channels exhibiting a constant shear rate throughout. F. Collagen type III-coated channels were incubated either statically or perfused at 1,000 s⁻¹ or 10,000 s⁻¹ with platelet poor plasma in the absence of SRG to allow VWF adhesion to collagen. Abciximab-treated, citrated whole blood, labelled with DiOC₆ was perfused over the differentially coated collagen III/VWF matrix as described in (B). Platelet rolling velocity on VWF / collagen III was assessed at SRGs (platelets rolling through the apex of the stenotic site) or constant shear (upstream of stenotic site) for all 3 coating regimens using particle tracking software. G. Abciximab-treated, citrated whole blood, labelled with DiOC₆ was perfused over a collagen III matrix, through 300 µm wide channels incorporating semi-circular stenotic segments which reduced the channel width to 60 µm. The diameters of semi-circular stenotic elements ranged from 600 µm and 1,000 µm to 2,000 µm. Whereas Δ shear of all three stenotic segments was equal, levels of elongational flow differed. H. Platelet rolling velocity on VWF-collagen III was assessed the same conditions as in (C) under SRGs (platelets rolling through the apex of the stenotic site) or constant shear (upstream of stenotic site) using particle tracking software. Data represented as mean±SEM; n=5; * p<0.05. (Dunnett’s multiple comparison one-way ANOVA).

FIG. 2. Platelet adhesion to collagen III is VWF-GPIb dependent. A. Blood, containing DiOC₆-labelled platelets, was treated with increasing concentrations of OS-1. Blood samples were drawn through collagen III coated channels at 1,500 s⁻¹. Fluorescence intensity was measured and expressed as relative platelet adhesion. B. Citrated whole blood treated with 20 µg ml⁻¹ Abciximab and 3 µM OS-1 was perfused through collagen III-coated microfluidics at constant shear rates ranging from 125 s⁻¹ to 2,000 s⁻¹. Adherent DiOC₆-labelled platelets were captured and automatically counted using ImageJ. C. Abciximab-treated (20 µg ml⁻¹), citrated whole blood, containing DiOC₆-labelled platelets, was treated with DAPT (30 µM aspirin plus 300 µM 2-MeSAMP) or control and drawn through stenotic or straight channels. Platelet rolling velocity on collagen III bound VWF was assessed under constant shear (upstream of stenotic site) (C) and at SRGs (in the apex of the stenosis).D. using particle tracking software. Rolling velocities were divided into three bins (0-3 µm s⁻¹; >3-6 µm s⁻¹; >6 µm s⁻¹) and presented as relative frequencies (C, D) Data represented as mean±SEM; n=5; * p<0.05 (multiple unpaired t-tests).

FIG. 3. Single-chain antibodies scFv A1 differentially inhibits VWF-platelet interaction at constant shear or gradients of shear. A. Abciximab-treated (20 µg ml⁻¹), citrated whole blood, labelled with 0.5 µg ml⁻¹ DiOC₆ containing scFv A1 (5 µg ml⁻¹) or control was perfused over a collagen III-coated surface as described in FIG. 1B. Platelet adhesion was assessed at the stenosis inlet, apex, outlet and upstream areas using particle tracking software. Data represented as matched individual data points or mean±SEM; n=5; * p<0.05. C. Relative VWF amount corrected for the level per platelet in platelet aggregates under SRGs and constant shear. Data represented as mean±SEM; n=3-4; (multiple t-test).

FIG. 4. scFv A1, but not OS-1 selectively inhibits thrombus formation in stenotic channel segments, a site of SRGs, but not at constant shear rate. Confocal microscopy images of platelet aggregates (A) and quantification (B) of aggregate growth. Citrated whole blood, labelled with 0.5 µg ml⁻¹ DiOC₆, containing 5 µg ml⁻¹ scFv A1 or control, was perfused over a collagen matrix for 10 minutes at an effective shear rate of 1,500 and 2,000 s⁻¹ in the stenosis apex (SRG) or upstream (constant shear). Note the inhibitory effect of scFv A1 at SRG (left panel) but not under constant shear (right panel). Data represented as mean±SEM; n=3-4; * p<0.05Confocal microscopy images of platelet aggregates (A) and quantification (B) of aggregate growth. Citrated whole blood, labelled with 0.5 µg ml⁻¹ DiOC₆, containing 5 µg ml⁻¹ scFv A1 or control, was perfused over a collagen matrix for 10 minutes at an effective shear rate of 1,500 and 2,000 s⁻¹ in the stenosis apex (SRG) or upstream (constant shear). Note the inhibitory effect of scFv A1 at SRG (left panel) but not under constant shear (right panel). Data represented as mean±SEM; n=3-4; * p<0.05. C. Confocal images of platelet aggregate formation as in (A) in the presence the GPlb inhibitor OS-1 (0.1 µg ml⁻¹) or control. D. Quantification of platelet aggregate growth after 15 minutes at sites of SRG or at constant shear in the presence of scFv A1, OS-1 or control (D). Data represented as mean±SEM; n=3-6; * p<0.05 (2-way ANOVA).

FIG. 5. scFv A1 specifically inhibits thrombus formation at sites of SRGs but not at constant shear rate. Citrated whole blood, labelled with 0.5 µg ml⁻¹ DiOC₆, containing 5 µg ml⁻¹ scFv A1 or control, was drawn at 1000 s⁻¹ through flow channels exhibiting a patch of perpendicularly-coated collagen type I (100 µg ml⁻¹) on the bottom surface, creating a BSA-collagen interface. A. Z-slices of confocal microscopy images were binarized and compiled to z-projections showing thrombus height for control (top panel) and scFv A1-loaded blood (bottom panel). B. Relative fluorescence intensities of platelet aggregates on sequential collagen patches in the same flow channel. C. Relative fluorescence intensities of platelet aggregates in the front-, centre- and rear areas of the collagen patch after 10 minutes of flow. D. CFD analysis of shear rates present at the surface of platelet aggregates in control blood (top panel) and scFv A1-treated blood (bottom panel). E. Graphical presentation of the platelet fluorescence distribution over the entire collagen patch at various timepoints. Data is mean of n=3 flows. F. Quantification of time-lapse confocal microscopy images showing reduced platelet deposition in the presence of scFv A1 at the front area but not the rear area of the patch. Data represented as mean±SEM; n=3; * p<0.05. G. Representative false colour images of platelet aggregate formation in the stenosis outlet region in the presence of (I) control mut-scFV and (II) 10 µg ml⁻¹ scFv A1 after 10 minutes of flow at 250 s⁻¹ input shear rate. H. Maximum surface coverage measured in the outlet analysis area as shown in G. Data represented as mean±SEM; n=3-5; *p<0.05 (multiple t-tests).

FIG. 6. Static assays to characterize binding of scFv A1 to VWF. A. Ristocetin (0.75 mg ml⁻¹) induced platelet agglutination traces. i) 300 µL washed platelets and 50 µL of platelet poor plasma (PPP) treated with 2, 4 µg ml⁻¹ scFv A1, Mutated scFv or Tyrode’s buffer. ii) 150 µL of isolated platelets, 50 µL of platelet poor plasma (PPP) treated with 40, 80 µg ml⁻¹ scFv A1, Mutated scFv or Tyrode’s buffer. iii) 50 µL of platelet poor plasma (PPP) preincubated with ristocetin for 30 min before mixing with 150 µL of isolated platelets, treated with 2.5 or 5 µg ml⁻¹ scFv A1, control scFv or Tyrode’s buffer. Agglutination was recorded for 10 minutes and normalized to Tyrode’s buffer sample. B. Western blot analysis of VWF protein (at 0.1, 10, 100 ng µl⁻¹ and 1 µg µl⁻¹) denatured via SDS-PAGE, probed with i) scFv-A1 (1 µg ml⁻¹) followed by anti-his HRP antibody (1:2500), ii) sheep α-VWF (1 µg ml⁻¹) antibody followed by donkey α-sheep HRP antibody (1:2500). C. ELISA absorbance values for binding of 5 µg ml⁻¹ scFv-1 A1 or control Mut-scFv to i) full length VWF (10 µg ml⁻¹) in the presence of 0.75 mg ml⁻¹ ristocetin, and ii) purified A1 domain (10 µg ml⁻¹). Data are mean ± standard error of mean (SEM); n=3. D. BLITZ association traces for scFv-A1 (20, 40 and 100 µg ml⁻¹) binding to VWF-A1 domain immobilized onto a protein-A BLITZ probe. Shown traces are background subtracted using a PBS only sample.

FIG. 7. VWF binding by scFv A1 and a commercially available antibody (Cablivi, Sanofi) in the presence or absence of ristocetin.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present invention is based on the surprising generation of an antibody that can bind to VWF in shear gradient conditions that occur during thrombus formation. An advantage of the antibodies of the invention is that they bind specifically to VWF in shear gradient conditions and do not bind to VWF in constant shear conditions. Therefore, the antibodies of the invention inhibit thrombosis without having a significant effect on haemostasis.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T.A. Brown (editor), Essential Molecular Biology: A Practical Approach, Volumes 1 and 2, IRL Press (1991), D.M. Glover and B.D. Hames (editors), DNA Cloning: A Practical Approach, Volumes 1-4, IRL Press (1995 and 1996), and F.M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J.E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901 -917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

Von Willebrand factor (VWF) is a glycoprotein that circulates in plasma as a series of multimers with sizes in the range of about 500-20000 kD. The multimeric form of VWF is composed of 250 kD polypeptide subunits joined together by disulfide bonds. VWF mediates early platelet adhesion to the subendothelium of the damaged vessel wall and only larger multimers exhibit hemostatic activity. Multimerized VWF binds to the platelet surface glycoprotein Gp1bα by interaction in the A1 domain of VWF to promote platelet adhesion. Endothelial cells secrete large polymeric forms of VWF, and it is presumed that these forms of VWF with low molecular weight (low molecular weight VWF) result from proteolytic cleavage. Multimers with large molecular weights accumulate in the Vibel-Parade body of endothelial cells and are released when stimulated.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject’s body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding protein” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens (e.g., VWF) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (~50 to 70 kD) covalently linked and two light chains (~23 kDa each). A light chain generally comprises a variable region (if present) and a constant domain and in mammals is either a k light chain or a λ light chain. A heavy chain generally comprises a variable region and one or two constant domain(s) linked by a hinge region to additional constant domain(s). Heavy chains of mammals are of one of the following types α, δ, ε, γ, or µ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C- terminus. The constant domain of the light chain (CL which is ~110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab’ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab’ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. Generally, but not necessarily, reference to binding means specific binding, and each term shall be understood to provide explicit support for the other term.

As used herein, the term “does not detectably bind” shall be understood to mean that an antigen binding protein, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilized and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore) in which the antigen binding protein is immobilized and contacted with an antigen.

As used herein, the term “epitope” (syn. “antigenic determinant”) shall be understood to mean a region of VWF to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three-dimensional structural characteristics, and/or specific charge characteristics.

As used herein, the term “subject” shall be taken to mean any animal including humans, for example a mammal. Exemplary subjects include but are not limited to humans and non-human primates. For example, the subject is a human.

### Antibodies

In one example, the antigen binding protein or VWF-binding protein as described herein according is an antibody. Any functional property of the antigen binding protein may be determined as described herein. For example, determining whether an antigen binding protein reduced or inhibits thrombus formation at shear gradient conditions but not constant shear conditions may be determined as outlined in the Examples, particularly Example 4. In particular, an antigen binding protein as described herein may inhibit thrombus formation and/or platelet deposition at shear rate gradients of 250-2,000 s⁻¹ or 187-1,500 s⁻¹ but not at constant shear conditions of 1,500 s⁻¹ or 2,000 s⁻¹.

Methods for generating antibodies are known in the art and/or described in Harlow and Lane (editors) Antibodies: A Laboratory Manual, Cold Spring Harbor Laboratory, (1988). Generally, in such methods VWF or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in US4196265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in US6300064 and/or US5885793. For example, the present inventors have isolated fully human antibodies from a phage display library.

The antibody of the present invention may be a synthetic antibody. For example, the antibody is a chimeric antibody, a humanized antibody, a human antibody synhumanized antibody, primatized antibody or a de-immunized antibody.

### Antibody Binding Domain Containing Proteins

### Single-Domain Antibodies

In some examples, a protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., US6248516).

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure VL-X-VH or VH-X-VL, wherein VL is an antibody light chain variable region, VH is an antibody heavy chain variable region, X is a linker comprising insufficient residues to permit the VH and VL in a single polypeptide chain to associate (or form an Fv) or is absent, and wherein the VH of one polypeptide chain binds to a VL of the other polypeptide chain to form an antigen binding domain, i.e., to form a Fv molecule capable of specifically binding to one or more antigens. The VL and VH can be the same in each polypeptide chain or the VL and VH can be different in each polypeptide chain so as to form a bispecific diabody (i.e., comprising two Fvs having different specificity).

### Single Chain Fv (scFv)

The skilled artisan will be aware that scFvs comprise VH and VL regions in a single polypeptide chain and a polypeptide linker between the VH and VL which enables the scFv to form the desired structure for antigen binding (i.e., for the VH and VL of the single polypeptide chain to associate with one another to form a Fv). For example, the linker comprises in excess of 12 amino acid residues with (Gly4Ser)3 being one of the more favored linkers for a scFv.

The present invention also contemplates a disulfide stabilized Fv (or diFv or dsFv), in which a single cysteine residue is introduced into a FR of VH and a FR of VL and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “VHH domains” in camelid antibodies and V-NAR in IgNAR, in order to distinguish them from the heavy chain variable regions that are present in conventional 4-chain antibodies (which are referred to as “VH domains”) and from the light chain variable regions that are present in conventional 4-chain antibodies (which are referred to as “VL domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

Other Antibodies and Proteins Comprising Antigen Binding Domains Thereof The present invention also contemplates other antibodies and proteins comprising antigen-binding domains thereof, such as:
(i) “key and hole” bispecific proteins as described in US5731168;
(ii) heteroconjugate proteins, e.g., as described in US4676980;
(iii) heteroconjugate proteins produced using a chemical cross-linker, e.g., as described in US4676980; and
(iv) Fab₃ (e.g., as described in EP19930302894).

### Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a VH or VL as described herein according to any example.

In another example, a nucleic acid of the invention comprises a sequence at least about 80% or 85% or 90% or 95% or 97% or 98% or 99% identical to a sequence encoding an antigen binding protein having a function as described herein according to any example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding protein described herein. A “moderate stringency” is defined herein as being a hybridization and/or washing carried out in 2 × SSC buffer, 0.1% (w/v) SDS at a temperature in the range 45° C. to 65° C., or equivalent conditions. A “high stringency” is defined herein as being a hybridization and/or wash carried out in 0.1 × SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tₘ) are known in the art. A temperature that is similar to (e.g., within 5° C. or within 10° C.) or equal to the Tₘ of a nucleic acid is considered to be high stringency. Medium stringency is to be considered to be within 10° C. to 20° C. or 10° C. to 15° C. of the calculated Tₘ of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., US4554101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized lgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S-S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall’Acqua et al., J Immunol. 177 : 1129-1138 2006; and/or Hezareh J Virol ;75: 12161-12168, 2001).

In another example, the Fc region is a chimeric Fc region, e.g., comprising at least one CH2 domain from an IgG4 antibody and at least one CH3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or US7083784.

### Protein Production

In one example, an antigen binding protein described herein according to any example is produced by culturing a hybridoma under conditions sufficient to produce the protein, e.g., as described herein and/or as is known in the art.

### Recombinant Expression

In another example, an antigen binding protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., US4816567 or US5530101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/ β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham’s Fl0 (Sigma), Minimal Essential Medium ((MEM), (Sigma), RPMI-1640 (Sigma), and Dulbecco’s Modified Eagle’s Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

It will be understood that the invention disclosed and defined in this specification extends to all alternative combinations of two or more of the individual features mentioned or evident from the text or drawings. All of these different combinations constitute various alternative aspects of the invention.

### Binding to VWF and Mutants Thereof

It will be apparent to the skilled artisan from the disclosure herein that antigen binding proteins of the present invention bind to VWF. Methods for assessing binding to a protein are known in the art, e.g., as described in Scopes (In: Protein purification: principles and practice, Third Edition, Springer Verlag, 1994). Such a method generally involves immobilizing the antigen binding protein and contacting it with labeled antigen (VWF). Following washing to remove non-specific bound protein, the amount of label and, as a consequence, bound antigen is detected. Of course, the antigen binding protein can be labeled and the antigen immobilized. Panning-type assays can also be used. Alternatively, or additionally, surface plasmon resonance assays can be used.

Optionally, the dissociation constant (Kd), association constant (Ka) and/or affinity constant (KD) of an immobilized antigen binding protein for VWF or an epitope thereof is determined. The “Kd” or “Ka” or “KD” for an VWF -binding protein is in one example measured by a radiolabeled or fluorescently-labeled VWF ligand binding assay. In the case of a “Kd”, this assay equilibrates the antigen binding protein with a minimal concentration of labeled VWF or epitope thereof in the presence of a titration series of unlabeled VWF. Following washing to remove unbound VWF or epitope thereof, the amount of label is determined, which is indicative of the Kd of the protein.

According to another example the Kd, Ka or KD is measured by using surface plasmon resonance assays, e.g., using BIAcore surface plasmon resonance (BIAcore, Inc., Piscataway, NJ) with immobilized VWF or a region thereof or immobilized antigen binding protein.

### Methods of Use

The antigen binding proteins of the invention may be administered to any subject in which inhibition of thrombosis would be beneficial. In particular, it is contemplated that the antigen binding proteins of the invention are particularly useful as antithrombotic agents or treating pathological thrombotic conditions. As used herein a pathological thrombotic condition may be one associated with, or caused by, the formation or presence of a thrombus or blood clot in a blood vessel. Exemplary pathological thrombotic conditions are those described herein.

Thrombosis is the occurrence of a clot in a blood vessel at a site of injury to the vessel or an inappropriate blood clot in a blood vessel and depends on the adhesion, activation, and aggregation of platelets. Red blood cells, whose function in thrombosis is not well defined, are especially abundant in venous thrombi. Final thrombus stability requires scaffolding provided by large polymers, such as fibrin and VWF.

As used herein, thrombosis refers to a thrombus (blood clot) inside a blood vessel. The term encompasses, without limitation, arterial and venous thrombosis, including deep vein thrombosis, portal vein thrombosis, jugular vein thrombosis, renal vein thrombosis, stroke, myocardial infarction, Budd-Chiari syndrome, Paget-Schroetter disease, and cerebral venous sinus thrombosis. Diseases and conditions associated with thrombosis and the risk of developing thrombosis or hypercoagulation include, without limitation, acute venous thrombosis, pulmonary embolism, thrombosis during pregnancy, hemorrhagic skin necrosis, acute or chronic disseminated intravascular coagulation (DIC), clot formation from surgery, long bed rest, long periods of immobilization, conditions that preclude or restrict movement such as partial or complete paralysis, morbid obesity, disorders that impede oxygen uptake and absorption such as lung disorders including lung cancer, COPD, emphysema, drug related fibrosis, cystic fibrosis, venous thrombosis, fulminant meningococcemia, acute thrombotic stroke, acute coronary occlusion, acute peripheral arterial occlusion, massive pulmonary embolism, axillary vein thrombosis, massive iliofemoral vein thrombosis, occluded arterial cannulae, occluded venous cannulae, cardiomyopathy, venoocclusive disease of the liver, hypotension, decreased cardiac output, decreased vascular resistance, pulmonary hypertension, diminished lung compliance, leukopenia, and thrombocytopenia.

The antigen binding proteins of the invention find particular application in treating pathological thrombotic conditions in a subject, wherein an effect on haemostasis should or needs to be avoided.

The antigen binding proteins of the invention are particularly useful as antithrombotic agents that do not compromise haemostasis, and as such are superior alternatives to traditional antithrombotic agents.

The term “haemostasis” as used herein refers to a coordinated mechanism that maintains the integrity of blood circulation following injury to the vascular system. In normal circulation without vascular injury, platelets are not activated and freely circulate. Vascular injury exposes sub-endothelial tissue to which platelets can adhere. Adherent platelets will attract other circulating platelets to form a preliminary plug that is particularly useful in closing a leak in a capillary or other small vessel. These events are termed primary haemostasis. This is, typically, rapidly followed by secondary haemostasis that involves a cascade of linked enzymatic reactions that result in plasma coagulation to reinforce the primary platelet plug.

Further, the present invention provides a method for inhibiting the growth of an existing thrombus in a subject, the method comprising administering an antigen binding protein of the invention as described herein, thereby inhibiting the growth of an existing thrombus in a subject. Preferably, the antigen binding proteins of the invention may be administered to a subject diagnosed at latter stages of atherothrombosis.

The present invention also provides a method of treating or reducing the risk of thrombosis in a patient at increased risk of developing thrombosis or hypercoagulation because of an underlying disease or treatment regimen. For example, some patients are at increased risk of thrombosis or hypercoagulation such as patients receiving transplanted cells, tissues or organs including hematopoietic transplants, bone marrow transplants, kidney, heart, liver, lung et al. as are patients receiving certain therapies such as chemotherapy or radiation.

In one embodiment, the antigen binding proteins of the invention may be used to treat conditions characterized by vascular occlusions, such as those that occur as a result of thrombus formation. Conditions that are characterized by vascular occlusions and justify treatment or prevention using antigen binding proteins of the invention include those that involve the arterial, capillary, and venous vasculature.

In case a stenosis in a vessel is detected via diagnostic imaging, the antibody would be administered systemically to reduce or prevent full occlusion causing organ damage.

In the setting of stroke, disease is sometimes caused by the rupture of atherosclerosis plaques in the carotid arteries. Vessel narrowing would be determined in the carotid arteries via ultrasound. Doppler ultrasound is able to detect the flow rate and shear in the vessel which could guide diagnosis.

In the setting of myocardial infarction, disease is caused by the rupture of atherosclerosis plaques in the coronary arteries. Vessel narrowing would be determined in the coronary arteries via X ray imaging with contrast agents (Iodine contrast). The degree of stenosis could indicate the flow rate and shear in the vessel which could guide diagnosis.

The same diagnostic imaging as well as follow up studies could determine the effectiveness of the antibody intervention in shear triggered thrombotic occlusion.

In the coronary arteries, occlusive thrombus formation often follows the rupture of atherosclerotic plaque. This occlusion is the major cause of acute myocardial infarction and unstable angina. Coronary occlusions can also occur following infections, inflammation, thrombolytic therapy, angioplasty, and graft placements. Similar principles apply to other parts of the arterial vasculature and include, among others, thrombus formation in the carotid arteries, which is the major cause of transient or permanent cerebral ischemia and stroke.

Venous thrombosis often follows stasis, infections, inflammatory reactions, and major surgery of the lower extremities or the abdominal area. Deep vein thrombosis results in reduced blood flow from the area distal to the thrombus and predisposes to pulmonary embolism. Pulmonary embolism is a major cause of post-surgical mortality. Disseminated intravascular coagulation (DIC) and acute respiratory distress syndrome (ARDS) where the antigen binding proteins of the invention are useful commonly occur within all vascular systems during bacterial sepsis, entry of foreign material into the blood stream following, e.g., trauma and child birth, immune reactions, inflammation, certain viral infections, certain platelet disorders, and cancer. Disseminated intravascular coagulation is a severe complication of many disease conditions and some drug treatments, including, for example, heparin. Thrombotic consumption of coagulation factors and platelets, and systemic coagulation results in the formation of life-threatening thrombi occurring throughout the microvasculature leading to local or widespread hypoxia and organ failure.

Thus, in one embodiment, a method is provided for inhibiting thrombosis in a subject in need thereof comprising administering to the subject an effective dose of an antigen binding proteins of the invention, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.

As described elsewhere herein, traditional antithrombotic agents are dangerous or even fatal when administered at their maximally effective doses. Accordingly, in another embodiment, a method is provided for reducing a required dose or complementing the effect of an antithrombotic agent in the treatment of thrombosis in a subject in need thereof comprising administering to the subject an effective dose of an antigen binding proteins of the invention. Traditional antithrombotic agents include direct or indirect thrombin inhibitor, a Factor X inhibitor, a Factor IX inhibitor, a Factor XII inhibitor, a Factor V inhibitor, a Factor VIII inhibitor, a Factor XIII inhibitor, a Factor VII inhibitor, a tissue factor inhibitor, a profibrinolytic agent, a fibrinolytic or fibrinogenolytic agent, a carboxypeptidase B inhibitor, a platelet inhibitor, a selective platelet count reducing agent, or a Factor XI inhibitor.

Direct thrombin inhibitors include argatroban and derivatives or analogs thereof, hirudin and recombinant or synthetic derivatives or analogs thereof, derivatives of the tripeptide Phe-Pro-Arg, chloromethylketone derivatives, ximelagatran and derivatives, metabolites, or analogs thereof, anion binding exosite inhibitors, and RNA/DNA aptamers.

Indirect thrombin inhibitors include heparin, coumarin, dermatan, and thrombomodulin.

TABLE 1

Summary of amino acid and nucleotide sequences

| | | | |
|---|---|---|---|
| Antibody ID | Region | SEQ ID NO: | Amino acid or nucleotide sequence |
| A1 | LCDR1 (protein; Kabat/Chothia) | 1 | KSVSTSGYSYMH |
| | LCDR2 (protein; Kabat/Chothia) | 2 | LVSNLES |
| | LCDR3 (protein; Kabat/Chothia) | 3 | QHIRELTRS |
| | HCDR1 (protein; Kabat) | 4 | NYWMS |
| | HCDR2 (protein; Kabat) | 5 | MIHPSDSDTRLNQKFKD |
| | HCDR3 (protein; Kabat) | 6 | FEGWLPV |
| | HCDR1 (protein; Chothia) | 7 | GYSFTNYWM |
| | HCDR2 (protein; Chothia) | 8 | HPSDSD |
| | HCDR3 (protein; Chothia | 9 | FEGWLPV |
| | VL (protein) | 10 | DIELTQSPASLAVSLGQSYRASCKSVSTSGYSYMHWNQQK |
| | | | PGQPPRLLIYLVSNLESGVPARFSGSGSGTDFTLNIHPVEE |
| | | | EDAATYYCQHIRELTRSEGDQAEINR |
| | VH (protein) | 11 | QVQLQQPGAELVRPGASVKLSCKASGYSFTNYWMSWVK |
| | | | QRPGQGLEWIGMIHPSDSDTRLNQKFKDKATLTVDKSSST |
| | | | AYMQLSSPTSEDSAGLLLCKKFEGWLPVLLGPRHHSHSLL |
| | | | S |
| | LCDR1 (DNA; Kabat/Chothia) | 12 | aagagcgtgtccacctccggctactcttacatg cac |
| | LCDR2 (DNA; Kabat/Chothia) | 13 | ctggtgtccaacctggaaagc |
| | LCDR3 (DNA; Kabat/Chothia) | 14 | cagcacatcagagagctgaccagaagc |
| | HCDR1 (DNA; Kabat) | 15 | aactactggatgagc |
| | HCDR2 (DNA; Kabat) | 16 | atgatccaccccagcgacagcgacacccggctgaaccagaagttcaaggac |
| | HCDR3 (DNA; Kabat) | 17 | ttcgagggctggctgcccgtg |
| | HCDR1 (DNA; Chothia) | 18 | ggctacagcttcaccaactactggatg |
| | HCDR2 (DNA; Chothia) | 19 | caccccagcgacagcgac |
| | HCDR3 (DNA; Chothia) | 20 | ttcgagggctggctgcccgtg |
| | VL (DNA) | 21 | GATATCGAGCTGACACAGAGCCCTGCCAGCCTGGCCGT |
| | | | GTCTCTGGGCCAGAGCTATAGAGCCAGCTGCAAGAGCG |
| | | | TGTCCACCTCCGGCTACTCTTACATGCACTGGAACCAGC |
| | | | AGAAGCCCGGCCAGCCCCCTAGACTGCTGATCTACCTG |
| | | | GTGTCCAACCTGGAAAGCGGCGTGCCCGCCAGATTTTC |
| | | | TGGCTCTGGCAGCGGCACCGACTTCACCCTGAACATCC |
| | | | ACCCCGTGGAAGAAGAGGACGCCGCCACCTACTACTGC |
| | | | CAGCACATCAGAGAGCTGACCAGAAGCGAGGGCGACCA |
| | | | GGCCGAGATCAATAGA |
| | VH (DNA) | 22 | CAGGTGCAGCTGCAGCAGCCTGGCGCTGAACTCGTGCG |
| | | | GCCAGGCGCTTCTGTGAAGCTGAGCTGTAAAGCCAGCG |
| | | | GCTACAGCTTCACCAACTACTGGATGAGCTGGGTCAAG |
| | | | CAGAGGCCAGGCCAGGGCCTGGAATGGATCGGCATGAT |
| | | | CCACCCCAGCGACAGCGACACCCGGCTGAACCAGAAGT |
| | | | TCAAGGACAAGGCCACCCTGACCGTGGACAAGAGCAGC |
| | | | AGCACCGCCTACATGCAGCTGTCCAGCCCCACCAGCGA |
| | | | GGATTCTGCTGGACTGCTGCTGTGCAAGAAGTTCGAGG |
| | | | GCTGGCTGCCCGTGCTGCTGGGACCTAGACACCACTCT |
| | | | CACAGCCTGCTGTCT |
| | LFR1 (protein; Kabat/Chothia) | 23 | DIELTQSPASLAVSLGQSYRASC |
| | LFR2 (protein; Kabat/Chothia) | 24 | WNQQKPGQPPRLLIY |
| | LFR3 (protein; Kabat/Chothia) | 25 | GVPARFSGSGSGTDFTLNIHPVEEEDAATYYC |
| | LFR4 (protein; Kabat/Chothia) | 26 | EGDQAEINR |
| | HFR1 (protein; Kabat) | 27 | QVQLQQPGAELVRPGASVKLSCKASGYSFT |
| | HFR2 (protein; Kabat) | 28 | WVKQRPGQGLEWIG |
| | HFR3 (protein; Kabat) | 29 | KATLTVDKSSSTAYMQLSSPTSEDSAGLLLCKK |
| | HFR4 (protein; Kabat) | 30 | LLGPRHHSHSLLS |
| | HFR1 (protein; Chothia) | 31 | QVQLQQPGAELVRPGASVKLSCKAS |
| | HFR2 (protein; Chothia) | 32 | SWVKQRPGQGLEWIGMI |
| | HFR3 (protein; Chothia) | 33 | TRLNQKFKDKATLTVDKSSSTAYMQLSSPTSEDSAGLLLCKK |
| | HFR4 (protein; Chothia) | 34 | LLGPRHHSHSLLS |
| | LFR1 (DNA; Kabat/Chothia) | 35 | gatatcgagctgacacagagccctgccagcctggccgtgtctctgggccagagct |
| | | | atagagccagctgc |
| | LFR2 (DNA; Kabat/Chothia) | 36 | tggaaccagcagaagcccggccagccccctagactgctgatctac |
| | LFR3 (DNA; Kabat/Chothia) | 37 | ggcgtgcccgccagattttctggctctggcagcggcaccgacttcaccctgaacatc |
| | | | caccccgtggaagaagaggacgccgccacctactactgc |
| | LFR4 (DNA; Kabat/Chothia) | 38 | gagggcgaccaggccgagatcaataga |
| | HFR1 (DNA; Kabat) | 39 | caggtgcagctgcagcagcctggcgctgaactcgtgcggccaggcgcttctgtga |
| | | | agctgagctgtaaagccagcggctacagcttcacc |
| | HFR2 (DNA; Kabat) | 40 | tgggtcaagcagaggccaggccagggcctggaatggatcggc |
| | HFR3 (DNA; Kabat) | 41 | aaggccaccctgaccgtggacaagagcagcagcaccgcctacatgcagctgtcc |
| | | | agccccaccagcgaggattctgctggactgctgctgtgcaagaag |
| | HFR4 (DNA; Kabat) | 42 | ctgctgggacctagacaccactctcacagcctgctgtct |
| | HFR1 (DNA; Chothia) | 43 | caggtgcagctgcagcagcctggcgctgaactcgtgcggccaggcgcttctgtga |
| | | | agctgagctgtaaagccagc |
| | HFR2 (DNA; Chothia) | 44 | agctgggtcaagcagaggccaggccagggcctggaatggatcggcatgatc |
| | HFR3 (DNA; Chothia) | 45 | acccggctgaaccagaagttcaaggacaaggccaccctgaccgtggacaagag |
| | | | cagcagcaccgcctacatgcagctgtccagccccaccagcgaggattctgctgga |
| | | | ctgctgctgtgcaagaag |
| | HFR4 (DNA; Chothia) | 46 | ctgctgggacctagacaccactctcacagcctgctgtct |
| VWF A1 domain | N/A | 47 | DISEPPLHDFYCSRLLDLVFLLDGSSRLSEAEFEVLKAFVVD |
| | | | MMERLRISQKWVRVAVVEYHDGSHAYIGLKDRKRPSELRR |
| | | | IASQVKYAGSQVASTSEVLKYTLFQIFSKIDRPEASRIALLLM |
| | | | ASQEPQRMSRNFVRYVQGLKKKKVIVIPVGIGPHANLKQIR |
| | | | LIEKQAPENKAFVLSSVDELEQQRDEIVSYLCDLAPEAPPPT |

### EXAMPLES

### Example 1 - Methods and Materials

### Single-Chain Antibody Generation

A panel of IgG antibodies was previously raised in mice against a 34/39 kDa fragment incorporating the human VWF A1 domain (De Luca et al. Blood. 2000 Jan 1;95(1):164-72). The sequence of the light and heavy chain was determined as described before (Stoll et al. Arteriosclerosis, thrombosis, and vascular biology. 2007 May;27(5):1206-12). Briefly, mRNA was prepared and reverse transcribed using an oligo-dT primer. The variable regions of the antibody’s heavy and light chain were amplified by polymerase chain reaction (PCR) using primers that anneal to conserved regions at the 5′ and 3′ ends of the variable regions and sequenced. The variable regions of the light and heavy chain of 2 antibodies from this panel were combined with a standard linker sequence and various tags added for purification and bioconjugation purposes (FLAG-tag, a HIS-tag and a sortase reaction site). The derived DNA-sequences were ordered from a commercial supplier (Geneart, Invitrogen) optimized for the final host expression system, cloned into a pSec-tag vector, expressed using a HEK293 cell line and purified as described before (Bonnard et al. J Am Heart Assoc. 2017 Feb 03;6(2); and Alt et al. Angew Chem Int Ed Engl. 2015 Jun 22;54(26):7515-9). In brief, after confirmation of the final clone, DNA was transfected with polyethylenimine (Polyscience Inc.) into human HEK293 kidney cells. After transfection, cells were cultured for 7 days at 37° C., with 5% CO₂, shaking at 140 rpm. Supernatant was collected and purified with a nickel-based metal affinity chromatography column, Ni-NTA column (Qiagen), according to the manufacturer’s instructions. Purified scFv was dialysed against PBS at 4° C. overnight. The cyclic peptide, OS-1 which is an allosteric inhibitor of GPlbα/VWF interactions with sequence CTERMALHNLC was obtained from Auspep (Parkville, Australia)

### Blood Sampling

The study was approved by Alfred Hospital ethics committee no 67/15 and all volunteers gave written consent. The volunteers abstained from aspirin, non-steroidal anti-inflammatory drugs (NSAIDs) and any other anti-platelet therapy for 10 days before blood was taken from the median cubital vein using a 21G butterfly needle and collected into vacuette tubes (Sarstedt AG & Co, Nurnbrecht, Germany) containing trisodium citrate (0.32% w/v final). Blood samples were immediately processed.

### Microfluidic Chips

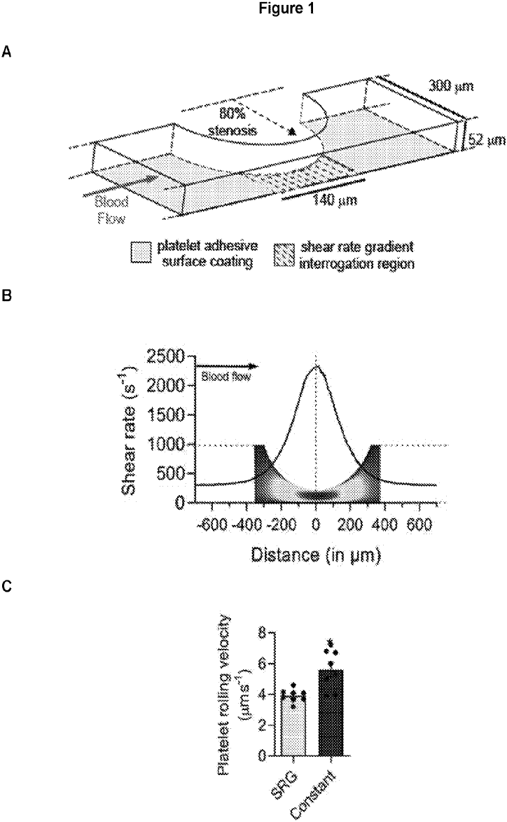

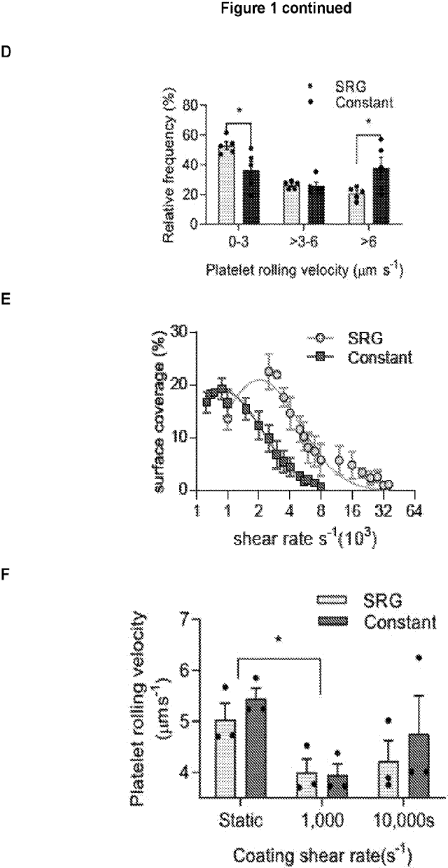

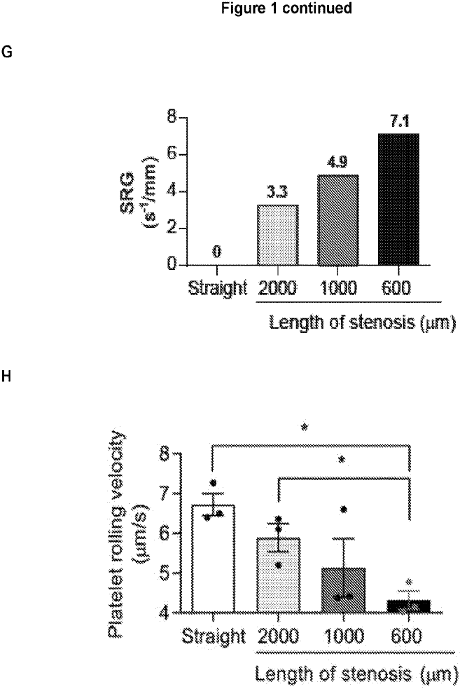

In-house designed PDMS microfluidic chips (FIG. 1A) contained channels of 52 µm height with a footprint of 300 µm x 46 mm (used in FIG. 1) or 300 µm x 17 mm. Selected channels incorporated a semi-circular stenotic section of various lengths (600 µm, 1000 µm or 2000 µm diameter), creating 80% lumen reduction. Chips were manufactured as previously reported (Westein et al. Proceedings of the National Academy of Sciences of the United States of America. 2013 Jan 22;110(4):1357-62). The produced PDMS chips were sealed with cover slips by hydrophobic interaction and coated with collagen by perfusion of collagen solution followed by static incubation at RT for at least 1 hr. Channels were rinsed with PBS and blocked with 2% BSA. Wall shear rates of the input flow for these microchannels were approximated from volumetric flow rates by using the equation, γ = 6Q/wh2. Herein, γ is the wall shear rate (s⁻¹), Q the volumetric flow rate (mls⁻¹), w the channel with (cm) and h the channel height (cm).

### Computational Fluid Dynamics and Calculations

Fluid dynamics were simulated with a COMSOL Multiphysics 4.2 laminar flow module applied to three-dimensional meshes. For all simulations, no-slip boundary conditions and Newtonian fluids were assumed. To obtain meshes from the plain channel geometries, channel dimensions were taken from the original computer-aided design (CAD) files and reconstructed in COMSOL. To obtain meshes from platelet aggregate geometries, confocal fluorescence microscopy image z-stacks of labeled platelets were processed in ImageJ. Each image in a z-stack was converted into a binary image by thresholding, and an x-y heightmap was constructed by summing all binary data in a z-stack followed by multiplying the result with the height of the original z-stack as documented by the confocal microscope. These heightmaps were imported in COMSOL to construct three-dimensional meshes. Input flow parameters were set based on pump settings as used in the experiments, and were adjusted to account for the reduced width of the fluorescence aggregate meshes compared to the full channel geometries. Both the computational fluid dynamical simulation and the calculations of fluid dynamical and geometrical parameters were carried out with the assumption that the channels and vessels were filled with Newtonian fluids. This assumption is valid because the shear rates are in a regime in which the non-Newtonian behavior of blood is limited.

### In Vitro Flow Perfusions

Citrated whole blood was incubated with DiOC₆ (0.5 µg ml⁻¹) (Sigma-Aldrich Co, St. Louis, MO, USA) and drawn through microfluidic channels by a programmable syringe pump (Legato 130, KD Scientific, USA). Time-lapse z-stack images of platelet deposition were captured in multiple track, resonant scanning mode, using a Nikon A1R confocal microscope system. Platelet deposition was recorded over time (2 frames per minute) for up to 20 minutes and quantified in ImageJ using a Z-projection of the acquired signal.

For experiments measuring rolling velocities, anti αllbβ3 antibody (abciximab; 20 µg ml⁻¹)-treated whole blood containing 0.5 µg ml⁻¹ DiOC₆ was drawn over a collagen type III-coated surface. Where indicated, blood was incubated with 30 µM aspirin (Sigma Aldrich, USA) and 300 µM 2-MeSAMP (Sigma-Aldrich Co, St. Louis, MO, USA) prior to blood perfusion. 10 second video clips of rolling platelets were recorded in the green fluorescent channel using an Olympus IX81 microscope system. Platelet rolling velocities were calculated using Diatrack v3.04 large scale particle tracking software (Vallotton et al. Microsc Microanal. 2013 Apr;19(2):451-60).

### Platelet Agglutination

Platelet-poor plasma (PPP; source of plasma VWF) was prepared by centrifugation of citrated whole blood at 300 × g for 10 min to obtain platelet rich plasma (PRP) and further centrifugation of the PRP fraction at 1700 × g for 7 min. Isolated platelets (3 × 10⁸/ml) suspended in platelet wash buffer (4.3 mM K₂HPO₄, 4.3 mM Na₂HPO₄, 24.3 mM NaH₂PO₄, 113 mM NaCl, 5.5 mM D-glu cose, and 10 mM theophylline, pH 6.5) were centrifuged at 1500 × g for 7 min. The washed platelets were subsequently resuspended at the same concentration in modified Tyrode’s buffer (10 mM Hepes, 12 mM NaHCO₃, 137 mM NaCl, 2.7 mM KCI and 5 mM glucose, pH 7.3) containing calcium (1 mM), magnesium (1 mM) and apyrase (0.02 U ml⁻¹) (ADPase activity). Isolated platelets (150, 300 µL at 3 × 10⁸/ml) and platelet poor plasma (50 µl) were incubated with 2, 2.5, 4, 5, 40 and 80 µg ml⁻¹ scFv A1 and the corresponding concentration of control scFv, or Tyrode’s buffer was used to neutralise any other aggregation mechanisms beside the VWF A1-GPlbα interaction. Platelet agglutination in response to 0.75 mg mL⁻¹ ristocetin (Helena Biosciences, Gateshead, UK) and in the presence of the αIIbβ3 (GPIIb/IIIa) blocker Integrilin was performed for 10 min on an AggRam system from Helena Laboratories, USA

### Western Blot

Denatured full length VWF (Mybiosource, USA) was electrophoresed at 0.1, 10, 100 ng and 1 µg using 5% SDS-Polyacrylamide gel at 125 V for 90 min. The protein was transferred onto PVDF membrane at 90V for 90 min followed by incubation with 1 µg ml⁻¹ scFv A1 overnight and the membrane was probed with horse-radish peroxidase (HRP)-conjugated detection antibody (1:2500, Sigma).

Later, the membrane was stripped and reprobed with 1 µg ml⁻¹ polyclonal sheep anti-VWF antibody (Abcam), followed by donkey anti-sheep HRP-conjugated secondary antibody (1:2500, R&D Systems, USA). Chemiluminescent detection of protein bands was done using ECL Western Blotting Substrate (ThermoFisher) and visualisation using a ChemiDoc Touch Imaging system (Bio-Rad, UK).

### Elisa

ELISA wells (Thermo Fisher Scientific) were coated with purified full length VWF or isolated A1 domain at 0.1, 1 and 10 µg ml⁻¹ in the presence of ristocetin (0.75 mg ml¹) and blocked with 2% BSA. Next, the coated wells were incubated with 5 µg ml⁻¹ of scFv A1 or sheep anti-VWF antibody followed by incubation with an HRP-conjugated secondary antibody (Anti-his HRP for scFv A1, Donkey anti-sheep HRP antibody for sheep anti-VWF antibody) (1:4,000). Readings were measured at 450 nm on a BMG Optima multi-well reader.

### Blitz

Bio-layer Interferometry was performed on a BIItz System (Pall Forte Bio, USA). Protein-A biosensors were hydrated for 30 minutes in PBS-Tween (0.05%) and loaded with VWF-A1 domain (130 µg ml⁻¹). Remaining binding sites were blocked by exposure to a polyclonal mouse IgG for 5 min. Sequential adhesion of scFv-A1 at 0, 20, 40 and 100 µg ml⁻¹ was monitored for 5 min per concentration. Real time visualisation of binding interactions between scFv A1 and full length VWF/A1 domain was performed using the BLItz Pro software. Traces were manually baseline aligned and corrected for control binding (0 µg ml⁻¹ scFv-A1).

### Example 2 - SRGs Activate VWF and Reduce Platelet Rolling Velocity on VWF/Collagen Type III

Exacerbated thrombus formation at atherosclerotic geometries is dependent on the degree of intraluminal sidewall protrusion which create SRGs. Thus, to elucidate the underlying mechanism of exacerbated thrombus formation under SRGs, platelet adhesion and VWF-activation were studied at sites of SRGs utilising a microfluidic platform. These channels exhibit a stenotic feature which resembled rheological parameters in larger carotid arteries in humans (i.e. slope of stenosis, wall shear rates at the inlet versus stenotic region, and maximal flow elongational rate) while keeping required blood volume low. Channels incorporated a semi-circular stenotic geometry of 600 µm in length to reduce the channel width by 80 % from 300 µm to 60 µm (FIG. 1A). Computational fluid dynamic analysis indicated that the wall shear rate was symmetrically distributed around the stenosis, with an 8-fold higher shear rate in the apex region compared with the pre-stenotic segment. At an input wall shear rate of 300 s⁻¹, platelets travelling through the stenosis are therefore exposed to an increase in shear rate to 2,400 s⁻¹ and a shear gradient (Δs⁻¹) of approximately 7.1 s⁻¹ µm⁻¹ followed by an identical shear decrease in the outlet of the stenosis (FIG. 1B).

Platelet rolling velocities, a measure of VWF-GPIbα bond strength, were reduced at SRGs (assessed in a 140 µm zone around the apex of the stenosis with a maximal shear rate of 2,400 s⁻¹) compared to matched constant wall shear rate of 2,400 s⁻¹ (3.9±0.2 µm s⁻¹ vs 5.4±0.7 µm s⁻¹) (FIG. 1C), with 52.9±2.5% of slow rolling platelets at SRGs compared to 36±5.7% at constant shear (FIG. 1D).

This finding indicates that shear rate gradients reduce platelet translocation, presumably through the unfolding of the VWF-A1 domain leading to stronger VWF-GPIb interactions. To confirm this finding, platelet surface coverage, a measure of VWF-platelet interaction was measured in stenotic channels over a wide range of input shear rates ranging from 125 s⁻¹ to 8,000 s⁻¹, resulting in shear rates of 1,000 s⁻¹ to 36,000 s⁻¹ in the stenosis apex and SRGs of approximately 3 s⁻¹ µm⁻¹ to 1000 s⁻¹ µm⁻¹. SRGs facilitated higher levels of transient platelet adhesion, with maximal platelet adhesion of 20.1±3.4% at 2,500 s⁻¹ and sustained platelet adhesion up to 7,000 s⁻¹, (7.89±1.86%; p=0.0462) while platelet adhesion rapidly decreased under constant shear from a maximum of 19.3±1.9% at 875 s⁻¹ and was abolished above 2,000 s⁻¹ (13.6±2.0%; p=0.0341) (FIG. 1E).

In order to elucidate the parameters critical for platelet-VWF interaction under SRG, VWF deposition onto collagen type III was tested under either static conditions or under flow in the absence of SRGs. Following VWF deposition onto collagen, the coated strips were overlaid with micro-channels featuring straight or stenotic sections. Platelet rolling velocities at SRGs or constant shear rate (CSR) were assessed at an effective wall shear rate of 2,400 s⁻¹. The rolling velocity of platelets on collagen strips that were statically incubated with VWF was higher (SRG: 5.03±0.31 µm s⁻¹; CSR: 5.44±0.2 µm s⁻¹) than those in channels incubated with VWF at a shear rate of 1,000 s⁻¹ (SRG: 4±0.26 µm s⁻¹; CSR: 3.95±0.21 µm s⁻¹, p<0.05) (FIG. 1F) indicating a role for flow in VWF deposition to collagen. However, when VWF was bound to collagen prior to platelet perfusion, the SRG specific increase of the GPIbα-VWF bond strength was lost (FIG. 1F), suggesting that VWF deposition needs to occur under real time SRGs in order to have subsequent increased engagement of GPIbα.

To test the effect of the steepness of the stenosis gradient on platelet rolling velocity, channels containing different stenotic segment lengths were manufactured. These ranged from 600 to 2000 µm in length, resulting in the same peak shear rate in the apex, but different shear rate gradients, ranging from 7.1 s⁻¹ µm⁻¹ to 3.2 s⁻¹ µm⁻¹ (FIG. 1G). Interestingly, platelet rolling velocities correlated with magnitude of SRGs. Rolling velocities were negatively correlated with the stenosis gradient, producing the slowest rolling velocity (4.32±0.23 µm s⁻¹) when travelling through a 600 µm stenosis and the highest (5.89±0.35 µm s⁻¹) when travelling through a 2,000 µm stenosis. However, the rolling velocity in the absence of SRG (i.e. under constant shear) was still higher (6.72±0.28 µm s⁻¹) (FIG. 1H).

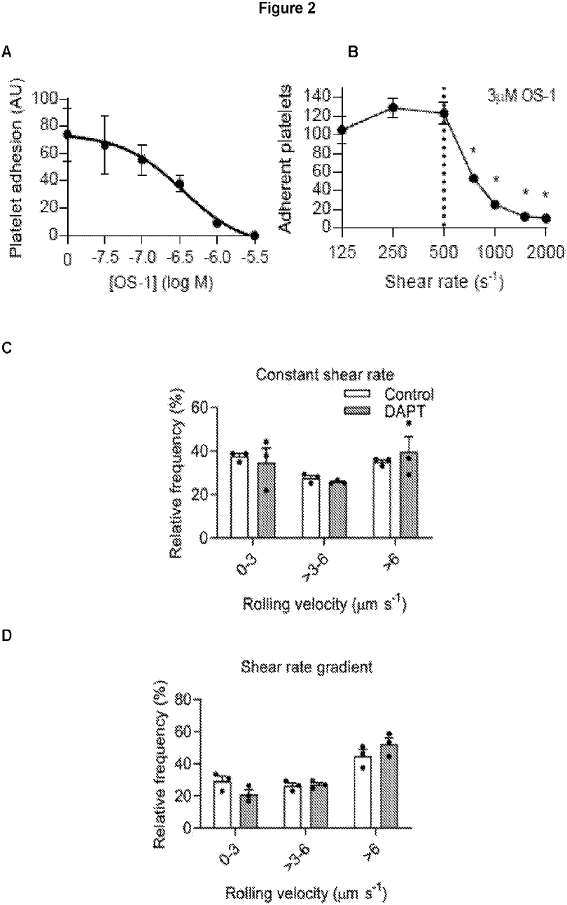

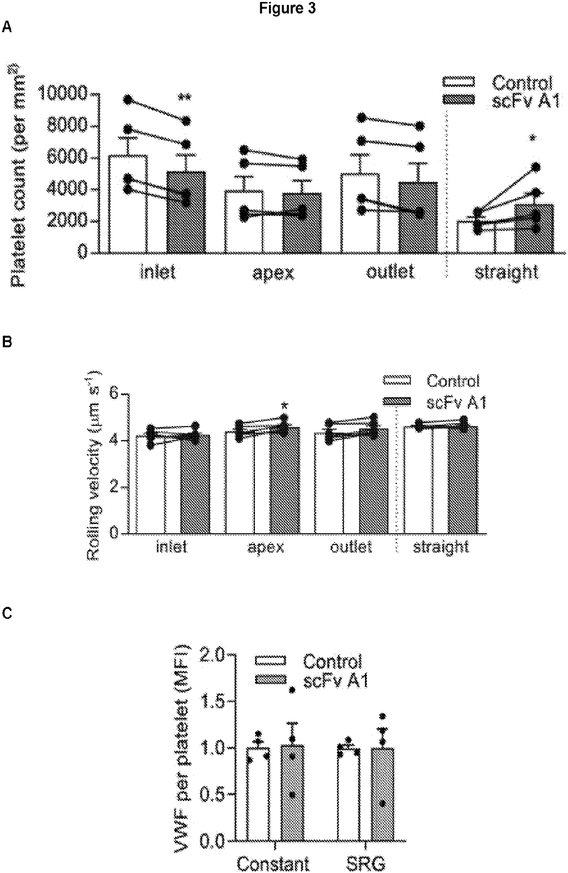

To confirm that previously observed platelet-surface interactions in our experimental setup were exclusively mediated through VWF-GPIbα, blood was incubated with the GPIbα inhibitor OS-1 (Benard et al. Biochemistry. 2008 Apr 22;47(16):4674-82). Blood perfusion at a shear rate of 1,500 s⁻¹ resulted in platelet adhesion of 74.2±19.5 AU which was concentration dependently reduced by OS-1. Platelet adhesion was completely abolished in the presence of 3 µM OS-1 (FIG. 2A). Furthermore, inhibition of platelet GPIbα by OS-1 (3 µM) caused a sharp decrease in numbers of adherent platelets from above 500 s⁻¹ (53±4 platelets per field) to 2,000 s⁻¹ (11±4 platelets per field) (FIG. 2B).

Having demonstrated that the observed interaction was GPIbα-VWF dependent with minor to zero contribution of direct collagen III interaction above 500 s⁻¹, the inventors tested whether platelet activation played a role in the observed differential rolling velocity. Thus, platelets were treated with aspirin plus the P2Y₁₂ inhibitor 2-MeSAMP. The drug treatment had no effect on rolling velocity at sites of SRG (FIG. 2D), nor at constant shear (FIG. 2C), highlighting that the observed rolling velocity on collagen III is solely determined by the biophysical interaction of GPIbα with VWF independent of platelet activation.

### Example 3 - Single-Chain Antibody A1 Specifically Inhibits SRGs-Dependent Platelet-VWF Interaction

SRGs have been shown to strongly activate VWF, which promotes platelet deposition at pathological shear rates thereby exacerbating thrombus formation. Specifically blocking SRG-mediated VWF-activation would potentially open up a new avenue of limiting excessive thrombus growth at the latter stages of atherothrombosis, thereby keeping the blood vessels patent.

To investigate whether SRG-activated VWF is a potential therapeutic target, the inventors produced several scFvs, based on the sequences of the variable heavy and light chain regions derived from a panel of mAbs raised against a 34/39 kDa VWF fragment incorporating the VWF A1 domain. The new scFvs were specifically tested for their capacity to inhibit VWF-platelet interactions selectively at sites of SRG. ScFv A1 reduced platelet-VWF adhesion specifically in the stenosis inlet, where shear gradients are the greatest from 6187±1097 platelets/mm² to 5158±1032 platelets/mm²; p=0.0013 (FIG. 3A). No differences in platelet adhesion were observed at the apex (3934±892 platelets/mm² control; 3805±783 platelets/mm² scFv A1, p=0.7932); nor in the stenosis outlet (5042±1167 platelets/mm² control; 4475±1199 platelets/mm² scFv A1; p=0.07). Surprisingly, scFv A1 showed an opposite effect in the straight section under constant shear, leading to increased platelet-deposition (2047±232 platelets/mm² control; 3096±690 platelets/mm² scFv A1; p=0.0195) (FIG. 3A). Platelet rolling velocities were largely unaffected by the addition of scFv A1 with the exception of the stenosis apex where a mild increase in rolling velocity was observed upon addition of scFv A1 (4.41±0.1 µm s⁻¹ control; 4.6±0.1 µm s⁻¹ scFv A1; p=0.038) (FIG. 3B). The relative amount of VWF per platelet deposited on aggregates in areas of SRGs and constant shear was not reduced by scFv A1 (FIG. 3C). Taken together these data suggest that scFv A1 interferes with a soluble fraction unfolded VWF which prevents VWF from engaging with surface bound platelets. Neither parent antibody CR1 nor CR2 from which the VH and VL, respectively, of A1 antibody were derived, exhibited the capacity to inhibit platelet adhesion under shear rate gradient and not inhibit under constant shear stress (data not shown).

### Example 4 - ScFv A1 Specifically Inhibits Shear Rate Gradient-Exacerbated Thrombus Formation

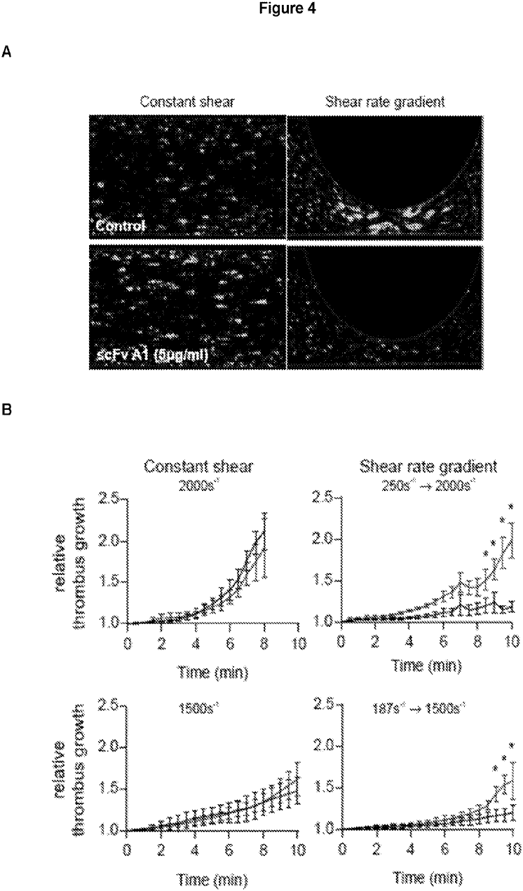

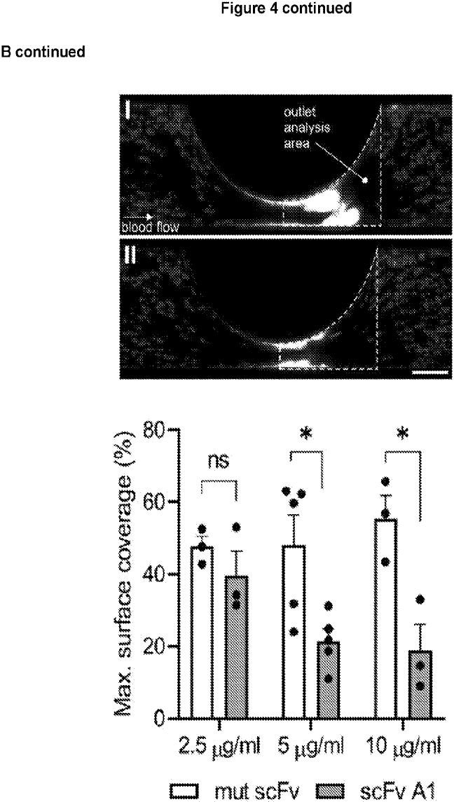

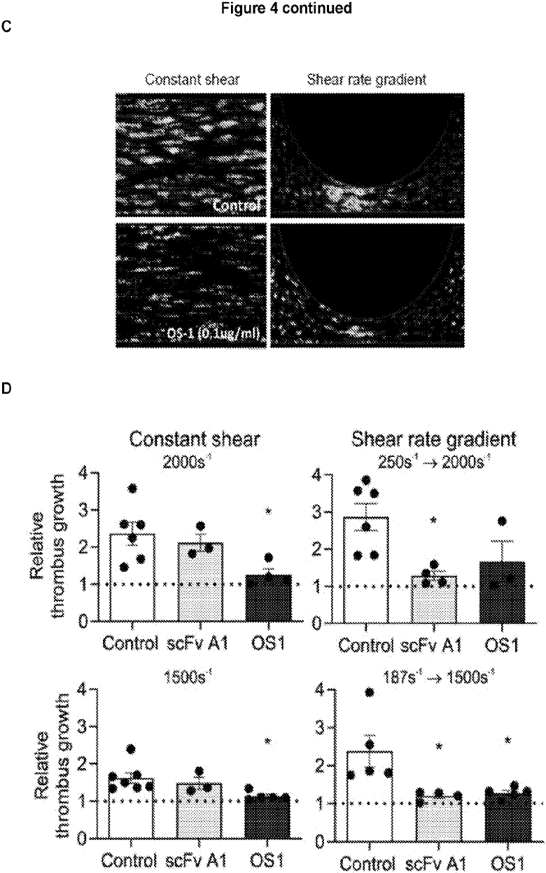

Taking the findings of the effect of scFv A1 on platelet rolling velocity and adhesion further into a more complex scenario, the inventors tested its capacity to specifically inhibit SRG-mediated thrombus formation. Blood containing scFv A1 (5 µg ml⁻¹) or control antibody was drawn through stenosis channels coated with collagen type I. ScFv A1 had no effect on platelet deposition at constant shear in the straight channel sections (FIG. 4A, left panel). Relative thrombus growth, defined as the ratio of intensity in the last frame over the first frame, ranged from 2.11±0.23 vs. 1.9±0.36 at 2,000 s⁻¹ to 1.48±0.16 vs. 1.62±0.2 at 1,500 s⁻¹ (FIG. 4B). However, under SRG conditions, scFv A1 significantly reduced thrombus formation from 1.85±0.28 to 1.17±0.07 (p<0.0001) at 2,000 s⁻¹ and from 1.58±0.22 to 1.2±0.09 (p<0.001) at 1,500 s⁻¹ (FIG. 4B, right panel). Inhibition of thrombus formation by scFv A1 was absent at a dose of 2.5 µg ml⁻¹ whereas 5 and 10 µg ml⁻¹ showed inhibition and to a similar degree. (FIG. 4B continued).

Next, the inhibitory effects of ScFv A1 were compared to the platelet GPIbα inhibitor OS-1 after 15 minutes of blood flow. Addition of 0.1 µM OS-1, a concentration that allowed residual platelet adhesion as determined in FIG. 2A, inhibited platelet deposition under both constant and shear gradient conditions (FIG. 4C). At 2000 s⁻¹ constant shear OS-1 reduced thrombus formation from while scFv A1 had no effect. A similar pattern, although weaker, was observed at 1,500 s⁻¹. In contrast, at SRGs (300-2,000 s⁻¹) and (187-1,500 s⁻¹), both OS-1 and scFv A1 inhibited thrombus formation. These data highlight the shear selective nature of ScFV A1 compared to a generic GPIb-VWF inhibitor.

### Example 5 - scFv A1 Does Not Interact With VWF Under Zero Shear Conditions

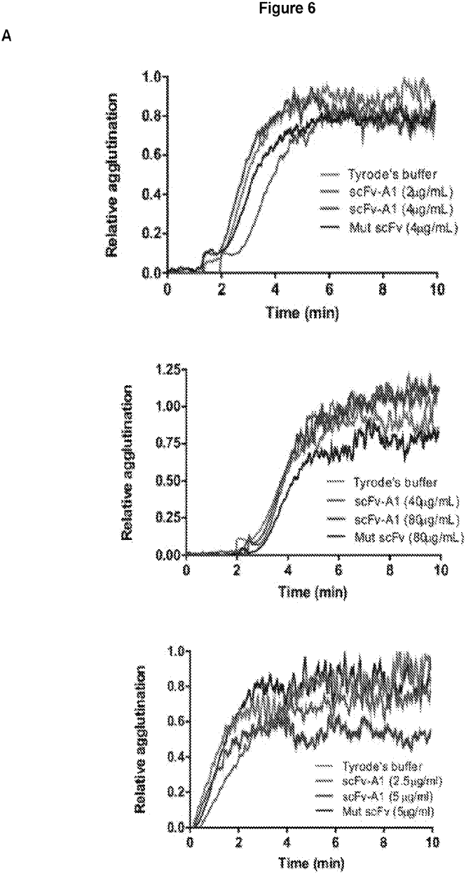

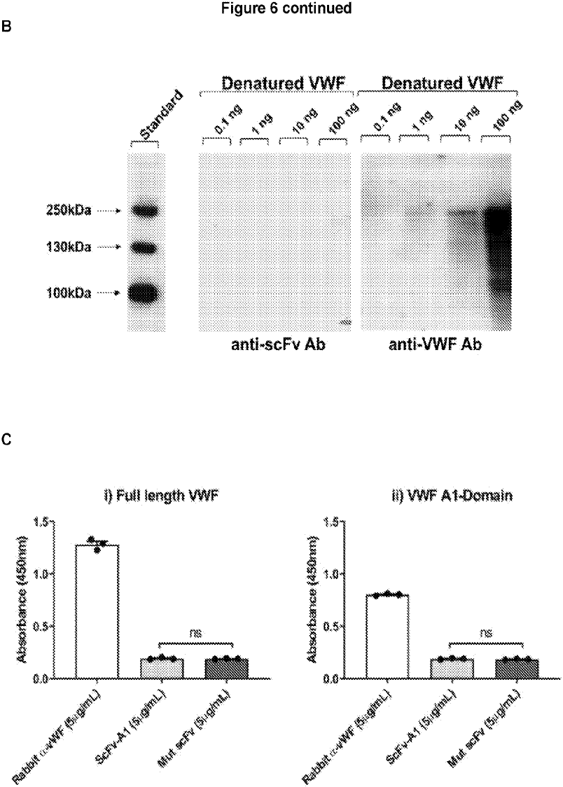

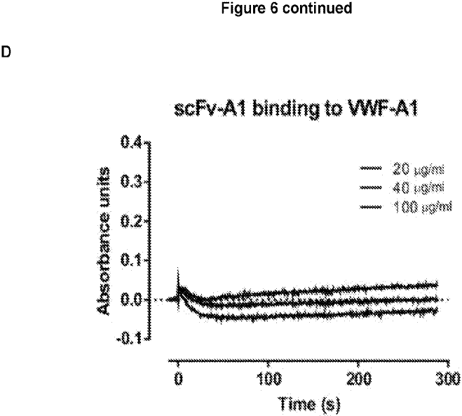

Next the inventors investigated whether scFv A1 could also inhibit VWF-GPIb binding under zero shear conditions. VWF dependent platelet agglutination was tested in a ristocetin-induced platelet agglutination test (RIPA). Since exposure of VWF to ristocetin or shear reportedly exposes similar epitopes within VWF, the inventors hypothesized that scFv A1 may inhibit ristocetin stimulated platelet agglutination. However, scFv A1 up to 80 µg ml⁻¹ (16 times higher than used in the microfluidic flow device) did not result in delayed or reduced agglutination of isolated platelets by ristocetin (0.75 mg ml⁻¹) in the presence of plasma VWF. However, when plasma VWF was preincubated with scFv A1 and ristocetin to facilitate their interaction prior to platelet binding, a minor inhibitory effect of scFv A1 on platelet agglutination was observed (FIG. 6A). Western blotting of denatured VWF with scFv A1 also did not show any complex formation (FIG. 6B). Similarly, scFv A1 did not bind to immobilized full length VWF or isolated VWF A1 domain in an ELISA test (FIG. 6C). Consistent with this result, the inventors also did not observe any binding between immobilized VWF-full length or VWF-A1 domain with scFv using the BLItz binding assay (FIG. 6D).

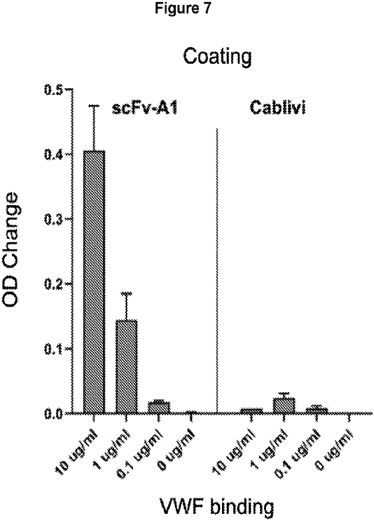

The inventors also compared the VWF binding by scFv A1 to that of a commercially available antibody (Cablivi®, Sanofi). Binding was assessed in the presence or absence of ristocetin (which mimics shear when interacting with VWF). The results, shown in FIG. 7, demonstrate that the mechanism of binding of scFv A1 differs from that of Cablivi, and in particular, the results demonstrate the conformational-dependent binding of scFv A1 (i.e., that scFv A1 binds to epitopes of VWF that are exposed in the shear conditions). In contrast, the binding by Cablivi to VWF is conformation independent.

### Example 6 - scFv A1 Dampens Thrombus Formation in the Latter Stages Only

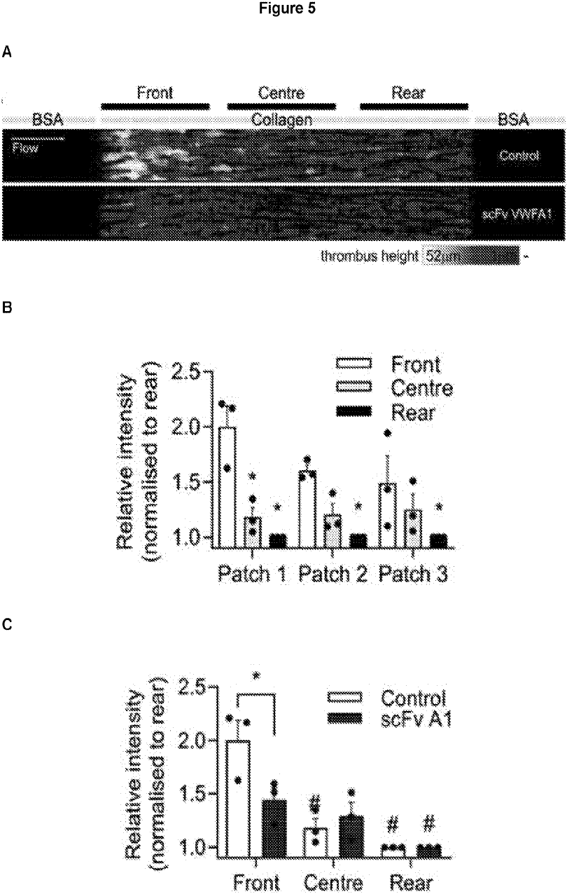

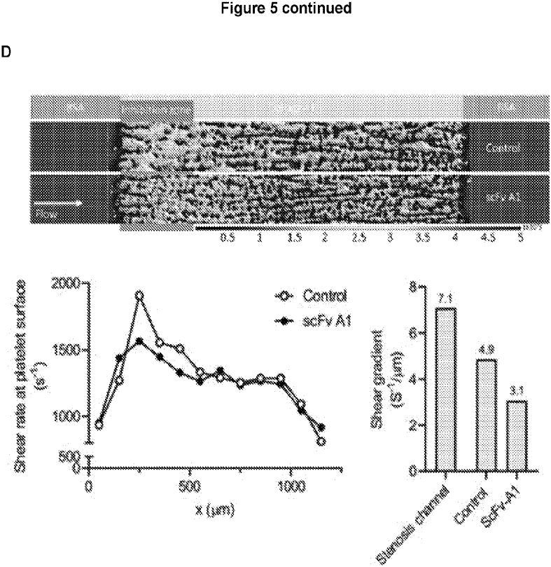

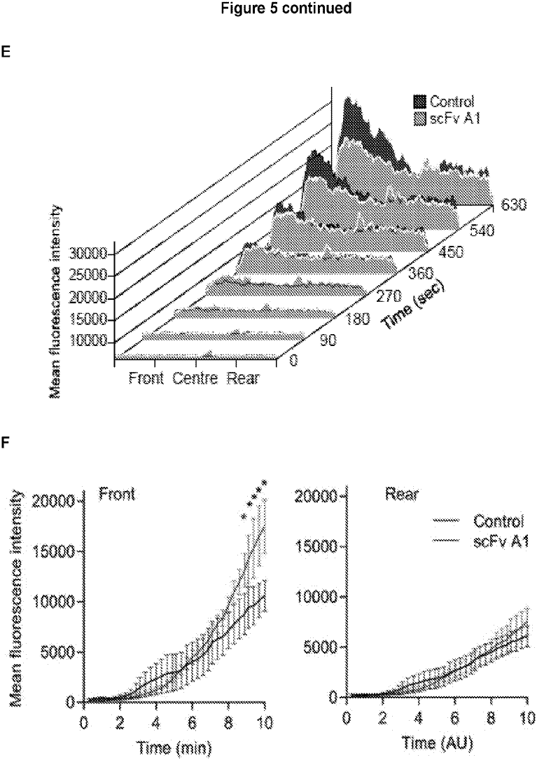

During the growth of an intraluminal thrombus, the local shear environment becomes progressively more complex. Here the inventors set out to mimic this highly dynamic process and characterize the inhibitory effects of scFv A1 within this process. Large platelet aggregates were allowed to form in straight channels and create their own local shear gradient environment during thrombus growth. Whole blood was perfused at 1,000 s⁻¹ over discrete patches of coated collagen type I surrounded by albumin coated areas (FIG. 5A). The presence of the albumin-collagen interface generates a sharp front of aggregates which grow to significant heights relative to the 52 µm high channels and as a result create a local steep shear gradient. After an initial phase of homogenous platelet accumulation across the entire collagen patch, platelet aggregation progressively increased at the front area of the patch (2.0±0.2-fold increase; front over rear) (FIG. 5B). This effect could not be explained by depletion of platelets at the boundary layer only as it was observed on sequential patches within a single channel. Next the inventors tested scFv A1 for its capacity to inhibit thrombus formation in this progressive shear gradient flow model. scFv A1 selectively attenuated platelet aggregation at the front area of the collagen patch (FIG. 5C) however, scFv A1 did not reduce platelet aggregation in the rear area where shear rates were constant and SRGs were absent. CFD analysis revealed that platelet aggregates at the front area of the patch experienced increased surface shear rates compared to those in the back area of the patch (FIG. 5D; upper panel), concomitant with platelet deposition patterns. As expected, scFv A1 reduced platelet deposition at the front area resulting in reduced aggregate surface shear rates (FIG. 5D; lower panel). The inhibitory effect of scFv A1 was observed even though the calculated maximum shear rate gradient, expressed as s⁻¹/µm, was lower in this flow model (4.9 and 3.1 s⁻¹/µm for control and scFv A1 respectively) compared to the SRGs calculated in the microfluidic stenosis channels used in FIGS. 1-4 (7.1 s⁻¹/µm) (FIG. 5D).

Time-lapse confocal microscopic analysis of platelet aggregate formation across the collagen patch revealed a lag phase of up to 180 seconds where small aggregates formed throughout the patch, followed by a growth phase at the front area of the patch where scFv A1 was inhibitory (FIG. 5E). Indeed, quantitative analysis showed that scFv A1 inhibited platelet aggregation only at the front of the patch but not at the rear (FIG. 5F).

In this study, the inventors demonstrate that SRGs exacerbate VWF-dependent platelet aggregation through increased VWF-GPIbα bond strength and that this process can selectively be inhibited using an antibody strategy that targets a SRG-sensitive epitope within the VWF A1-domain. Thus, SRGs are a potential novel drug-target for the prevention of occlusive thrombus formation.

The blood flow dynamics in areas of flow restriction, stenosis or mural thrombus formation have long been known to create gradients in shear rate, or SRGs. The inventors have shown exacerbated thrombus formation in the outlet of stenotic vessel sections to be VWF dependent. While high constant shear in the range of approximately 5,000 s⁻¹ to 8,000 s⁻¹ is required to unfold and thereby activate VWF, SRGs dramatically reduce this shear range by 10-fold. This means SRG mediated platelet aggregation can occur in virtually all vessel beds where a flow constriction occurs.

First, the inventors investigated the effects of shear rate gradients on platelet deposition. The inventors monitored the platelet-surface interaction on a collagen type III matrix because this type of collagen shows high affinity for VWF, resulting in high VWF density, while causing mild platelet activation. To prevent engagement of αIIbβ3 with VWF as well as platelet aggregation, whole blood was preincubated with the αIIbβ3-inhibitor abciximab. Most importantly, the use of collagen III as a physiological VWF substrate ensured correct conformation of shear-immobilised VWF. Recording and tracking individual platelets rolling on VWF bound to a collagen type III matrix either through a stenotic section (i.e. exposed to SRG) or through a straight section with constant shear, enabled analyse rolling velocities under different shear conditions, i.e. measure the effect of SRGs on VWF-platelet bond strength. While platelet rolling is typically not observed in vivo and therefore has limited physiological relevance, it is a sensitive readout reporting on VWF-GPIbα bond strength.

The data herein show that rolling velocities at SRGs are lower than at matched constant shear, suggesting that SRGs at the inlet of the stenosis, lead to rapid unfolding of VWF beyond the degree expected under matched constant shear. This unfolding promotes the formation of catch bonds between VWF and GPIbα and reduces the rolling velocity of platelets on the adhesive surface either due to a higher degree of unfolding per VWF molecule or more unfolded VWF molecules in general. In analogy to tether formation in platelets, this feature of SRGs may arrest a platelet sufficiently long to facilitate firm adhesion to the adhesive surface or a neighbouring platelet, thereby exacerbating thrombus formation under SRGs.

The inventors included the combination of common antiplatelet drugs – aspirin plus a P2Y₁₂ inhibitor – in their experiments elucidating the differences in rolling velocity between SRG and constant shear (FIGS. 2C, D). These drugs did not prevent the pro-adhesive phenotype of platelets under SRG conditions in these experiments, highlighting that platelet activation does not play a key role in SRG mediated platelet adhesion and that current antiplatelet therapy would be unable to target initial platelet tethering or mechanical hyper-activation of VWF by SRGs which culminates in exacerbated thrombosis.

Others have identified the GPIbα-VWF axis as a potential target and various drug candidates are currently under investigation. In contrast to our SRG-specific approach, these drugs take a systemic approach, thereby potentially affecting haemostasis. The inventors sought an approach that would allow inhibition of VWF specifically at sites of SRGs, a feature of atherothrombosis, without affecting VWF-activation under constant shear, which prevales during normal haemostasis.

The blocking strategy contained the novel scFv A1, which was based on the sequences from a previously developed human mAB panel raised in mice against a 39/34-kDa VWF fragment (Leu-480/Val-481-Gly-718) which encompasses the A1 domain. The scFvs are an emerging class of small recombinant antibodies that are economical to produce and suitable for therapeutic applications as they are less immunogenic than full-length monoclonal antibodies. The basis for the generation of the single chain antibody scFv A1 involved combining the variable heavy chain of mAb CR1 which recognizes a linear epitope in VWF-A1 with the variable light chain of mAb CR2 which recognises a conformation specific epitope in the VWF A1 domain.

Assessing single platelet binding to the adhesive surface, the novel single-chain antibody A1 showed selective platelet-VWF inhibition at stenotic sites where SRGs are prevalent compared to areas of constant shear. Specifically, platelet adhesion to the collagen III matrix was blocked in the stenosis inlet, the zone exhibiting the greatest shear gradient. The platelet rolling velocity in the presence of scFv A1 was increased in the stenosis but not upstream or downstream form the stenosis, suggesting that the scFv A1 did not systemically block the VWF-GPIbα interaction but rather specifically at the stenosis and in a transient manner. Similarly, under constant shear conditions platelet aggregation on collagen bound VWF in the presence of scFv A1 was not inhibited. In contrast to the SRG sensitive targeting of scFv A1 to VWF, the GPIbα/VWF inhibitor OS-1 inhibited platelet aggregation across all shear types and was not specific to SRG-mediated platelet aggregation. Taken together, these results suggest that scFv A1 targets the transiently present hyperactive form of VWF at the site of a stenosis.

The activation state of VWF during the flow immobilization to collagen or immobilized platelets appeared to modulate the subsequent recruitment of additional platelets. When VWF was deposited onto collagen prior to the flow experiment, SRGs did not exert this pro-adhesive effect on platelets, indicating that the activation state of VWF in the fluid phase at the moment of deposition onto collagen or immobilized platelets is a key determinant in the pro-thrombotic phenotype of SRGs.

VWF unfolding in an accelerating flow field is likely to be very transient, with apparent rapid refolding in areas downstream of SRGs where shear rates resume a constant level again. Therefore, the binding of scFv A1 to VWF is an elusive event that could only be achieved under shear gradient forces in the microfluidic flow assays.

Indeed, the inventors were unable to establish evidence of scFv A1 binding to VWF in other assays with zero shear conditions, likely due to the epitope for scFv A1 being shielded or not in a correct conformation. Since SRG are thought to be mechanistically similar to very high constant shear, we aimed to mimic the shear unfolding of VWF with the biochemical modulator ristocetin. This antibiotic is known to modulate the VWF-GPIb interaction, binding similar epitopes to those exposed by elevated shear. After optimizing experimental conditions for platelet agglutination, scFv A1 inhibited ristocetin-induced platelet agglutination in a minor way suggesting that ristocetin may expose epitopes in VWF that are recognized by scFv A1.

In conclusion, the inventors have shown that pro-thrombotic effects of SRGs, which lead to “hyperactivation” of VWF can be site specifically inhibited with the scFv A1 antibody without interfering with the VWF-GPIbα interaction under normal flow. This targeting strategy therefore has direct application in antithrombotic therapy which mechanically decouples thrombosis from haemostasis and therefore does not contribute to increased bleeding tendency, one of the main culprits in current antiplatelet drug development.

It will be understood that the invention disclosed and defined in this specification extends to all alternative combinations of two or more of the individual features mentioned or evident from the text or drawings. All of these different combinations constitute various alternative aspects of the invention.

1. An antigen binding protein comprising an antigen binding domain that binds to Von Willebrand factor (VWF) under shear gradient conditions.

2. An antigen binding protein according to claim 1, wherein the antigen binding protein binds to the A1 domain.

3. An antigen binding domain of claim 1 or 2, wherein the antigen binding protein of the invention reduces platelet-VWF interaction under shear gradient conditions.

4. An antigen binding protein according to any one of claims 1 to 3, wherein the antigen binding protein competitively inhibits binding of the A1 antibody comprising a VH comprising a sequence set forth in SEQ ID NO: 11 and a VL comprising a sequence set forth in SEQ ID NO: 10.

5. An antigen binding protein according to any one of claims 1 to 4, wherein the antigen binding protein binds to the same epitope on VWF as an antibody that comprises a variable heavy chain (VH) domain comprising the amino acid sequence as set forth in SEQ ID NO: 11 and a variable light chain (VL) domain comprising the amino acid sequence as set forth in SEQ ID NO: 10.

6. An antigen binding protein according to any one of claims 1 to 5, wherein the antigen binding domain comprises a VH comprising a CDR1, CDR2 and CDR3 as set forth in SEQ ID NO: 11, and a VL comprising a CDR1, CDR2 and CDR3 as set forth in SEQ ID NO: 10.

7. An antigen binding protein according to any one of claims 1 to 6, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4, a CDR2 comprising a sequence at least about 80%, at least 85%, at least 90%, at least 92%, at least 95%, at least 97%, at least 99% identical to a sequence set forth in SEQ ID NO:5 and a CDR3 comprising a sequence at least about 80%, at least 85%, at least 90%, at least 92%, at least 95%, at least 97%, at least 99% identical to a sequence set forth in SEQ ID NO: 6;
(ii) a VH comprising a sequence at least about 95% or 96% or 97% or 98% or 99% identical to a sequence set forth in SEQ ID NO: 11;
(iii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(iv) a VL comprising a sequence at least about 95% or 96% or 97% or 98% or 99% identical to a sequence set forth in SEQ ID NO: 10;
(v) a VH comprising a CDR1 comprising a sequence set forth in SEQ ID NO: 4, a CDR2 comprising a sequence set forth in SEQ ID NO: 5 and a CDR3 comprising a sequence set forth in SEQ ID NO: 6;
(vi) a VH comprising a sequence set forth in SEQ ID NO: 11;
(vii) a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3;
(viii) a VL comprising a sequence set forth in SEQ ID NO: 10;
(ix) a VH comprising a CDR1 comprising a sequence set forth in SEQ ID NO: 4, a CDR2 comprising a sequence set forth in SEQ ID NO: 5 and a CDR3 comprising a sequence set forth in SEQ ID NO: 6; and a VL comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3; or
(x) a VH comprising a sequence set forth in SEQ ID NO: 11 and a VL comprising a sequence set forth in SEQ ID NO: 10.

8. An antigen binding protein according to claim 7, wherein the antigen binding domain comprises:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4, a CDR2 comprising a sequence at least about 80%, at least 85%, at least 90%, at least 92%, at least 95%, at least 97%, at least 99% identical to a sequence set forth in SEQ ID NO:5 and a CDR3 comprising a sequence at least about 80%, at least 85%, at least 90%, at least 92%, at least 95%, at least 97%, at least 99% identical to a sequence set forth in SEQ ID NO: 6; and
(ii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3; Or
(iii) a VH comprising a sequence at least about 95% or 96% or 97% or 98% or 99% identical to a sequence set forth in SEQ ID NO: 11; and
(iv) a VL comprising a sequence at least about 95% or 96% or 97% or 98% or 99% identical to a sequence set forth in SEQ ID NO: 10; Or
(v) a VH comprising a CDR1 comprising a sequence set forth in SEQ ID NO: 4, a CDR2 comprising a sequence set forth in SEQ ID NO: 5 and a CDR3 comprising a sequence set forth in SEQ ID NO: 6; and
(vi) a VL comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3; Or
(vii) a VH comprising a sequence set forth in SEQ ID NO: 11 and a VL comprising a sequence set forth in SEQ ID NO: 10.

9. An antigen binding protein according to claim 7 or 8, wherein the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO:27, a FR2 comprising a sequence at least about 80%, at least 85%, at least 90%, at least 92%, at least 95%, at least 97%, at least 99% identical to a sequence set forth in SEQ ID NO:28, a FR3 comprising a sequence at least about 80%, at least 85%, at least 90%, at least 92%, at least 95%, at least 97%, at least 99% identical to a sequence set forth in SEQ ID NO: 29, and a FR4 comprising a sequence at least about 80%, at least 85%, at least 90%, at least 92%, at least 95%, at least 97%, at least 99% identical to a sequence set forth in SEQ ID NO: 30;
(ii) a VL comprising a FR1 comprising a sequence at least about 80%, at least 85%, at least 90%, at least 92%, at least 95%, at least 97%, at least 99% identical to a sequence set forth in SEQ ID NO: 23, a FR2 comprising a sequence at least about 80%, at least 85%, at least 90%, at least 92%, at least 95%, at least 97%, at least 99% identical to a sequence set forth in SEQ ID NO: 24, a FR3 comprising a sequence at least about 80%, at least 85%, at least 90%, at least 92%, at least 95%, at least 97%, at least 99% identical to a sequence set forth in SEQ ID NO: 25, and a FR4 comprising a sequence at least about 80%, at least 85%, at least 90%, at least 92%, at least 95%, at least 97%, at least 99% identical to a sequence set forth in SEQ ID NO: 26;
(iii) a VH comprising a FR1 comprising a sequence set forth in SEQ ID NO: 27, a FR2 comprising a sequence set forth in SEQ ID NO: 28, a FR3 comprising a sequence set forth in SEQ ID NO: 29, and a FR4 comprising a sequence set forth in SEQ ID NO: 30;
(iv) a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26; or
(v) a VH comprising a FR1 comprising a sequence set forth in SEQ ID NO: 27, a FR2 comprising a sequence set forth in SEQ ID NO: 28, a FR3 comprising a sequence set forth in SEQ ID NO: 29, and a FR4 comprising a sequence set forth in SEQ ID NO: 30; and a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26.

10. An antigen binding protein according to any one of claims 1 to 6, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:7, a CDR2 comprising a sequence at least about 80%, at least 85%, at least 90%, at least 92%, at least 95%, at least 97%, at least 99% identical to a sequence set in SEQ ID NO: 8 and a CDR3 comprising a sequence at least about 80%, at least 85%, at least 90%, at least 92%, at least 95%, at least 97%, at least 99% identical to a sequence set forth in SEQ ID NO: 9;
(ii) a VH comprising a sequence at least about 95% or 96% or 97% or 98% or 99% identical to a sequence set forth in SEQ ID NO: 11;
(iii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(iv) a VL comprising a sequence at least about 95% or 96% or 97% or 98% or 99% identical to a sequence set forth in SEQ ID NO: 10;
(v) a VH comprising a CDR1 comprising a sequence set forth in SEQ ID NO: 7, a CDR2 comprising a sequence set forth in SEQ ID NO: 8 and a CDR3 comprising a sequence set forth in SEQ ID NO: 9;
(vi) a VH comprising a sequence set forth in SEQ ID NO: 11;
(vii) a VL comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3;
(viii) a VL comprising a sequence set forth in SEQ ID NO: 10;
(ix) a VH comprising a CDR1 comprising a sequence set forth in SEQ ID NO: 7, a CDR2 comprising a sequence set forth in SEQ ID NO: 8 and a CDR3 comprising a sequence set forth in SEQ ID NO: 9; and a VL comprising a CDR1 comprising a sequence set forth in SEQ ID NO: 1, a CDR2 comprising a sequence set forth in SEQ ID NO: 2 and a CDR3 comprising a sequence set forth in SEQ ID NO: 3; or
(x) a VH comprising a sequence set forth in SEQ ID NO: 11 and a VL comprising a sequence set forth in SEQ ID NO: 10.

11. An antigen binding protein according to any one of claims 1 to 6, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:7, a CDR2 comprising a sequence at least about 80%, at least 85%, at least 90%, at least 92%, at least 95%, at least 97%, at least 99% identical to a sequence set in SEQ ID NO:8 and a CDR3 comprising a sequence at least about 80%, at least 85%, at least 90%, at least 92%, at least 95%, at least 97%, at least 99% identical to a sequence set forth in SEQ ID NO: 9; and
(ii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3; Or
(iii) a VH comprising a sequence at least about 95% or 96% or 97% or 98% or 99% identical to a sequence set forth in SEQ ID NO: 11; and
(iv) a VL comprising a sequence at least about 95% or 96% or 97% or 98% or 99% identical to a sequence set forth in SEQ ID NO: 10; Or
(v) a VH comprising a CDR1 comprising a sequence set forth in SEQ ID NO: 7, a CDR2 comprising a sequence set forth in SEQ ID NO: 8 and a CDR3 comprising a sequence set forth in SEQ ID NO: 9; and
(vi) a VL comprising a CDR1 comprising a sequence set SEQ ID NO: 1, a CDR2 comprising a sequence set forth in SEQ ID NO: 2 and a CDR3 comprising a sequence set forth in SEQ ID NO: 3; Or
(vii) a VH comprising a sequence set forth in SEQ ID NO: 11 and a VL comprising a sequence set forth in SEQ ID NO: 10.

12. An antigen binding protein according to claim 10 or 11, wherein the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in SEQ ID NO: 31, a FR2 comprising a sequence at least about 80%, at least 85%, at least 90%, at least 92%, at least 95%, at least 97%, at least 99% identical to a sequence set forth in SEQ ID NO: 32, a FR3 comprising a sequence at least about 80%, at least 85%, at least 90%, at least 92%, at least 95%, at least 97%, at least 99% identical to a sequence set forth in SEQ ID NO: 33, and a FR4 comprising a sequence at least about 80%, at least 85%, at least 90%, at least 92%, at least 95%, at least 97%, at least 99% identical to a sequence set forth in SEQ ID NO: 34;
(ii) a VL comprising a FR1 comprising a sequence at least about 80%, at least 85%, at least 90%, at least 92%, at least 95%, at least 97%, at least 99% identical to a sequence set forth in SEQ ID NO: 23, a FR2 comprising a sequence at least about 80%, at least 85%, at least 90%, at least 92%, at least 95%, at least 97%, at least 99% identical to a sequence set forth in SEQ ID NO: 24, a FR3 comprising a sequence at least about 80%, at least 85%, at least 90%, at least 92%, at least 95%, at least 97%, at least 99% identical to a sequence set forth in SEQ ID NO: 25, and a FR4 comprising a sequence at least about 80%, at least 85%, at least 90%, at least 92%, at least 95%, at least 97%, at least 99% identical to a sequence set forth in SEQ ID NO: 26;
(iii) a VH comprising a FR1 comprising a sequence set forth in SEQ ID NO: 31, a FR2 comprising a sequence set forth in SEQ ID NO: 32, a FR3 comprising a sequence set forth in SEQ ID NO: 33, and a FR4 comprising a sequence set forth in SEQ ID NO: 34;
(iv) a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26; or
(v) a VH comprising a FR1 comprising a sequence set forth in SEQ ID NO: 31, a FR2 comprising a sequence set forth in SEQ ID NO: 32, a FR3 comprising a sequence set forth in SEQ ID NO: 33, and a FR4 comprising a sequence set forth in SEQ ID NO: 34; and a VL comprising a FR1 comprising a sequence set forth in SEQ ID NO: 23, a FR2 comprising a sequence set forth in SEQ ID NO: 24, a FR3 comprising a sequence set forth in SEQ ID NO: 25, and a FR4 comprising a sequence set forth in SEQ ID NO: 26.

13. An antigen binding protein according to any one of claims 1 to 12, wherein the antigen binding protein is in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to an immune effector cell.

14. An antigen binding protein according to any one of claims 1 to 12, wherein the antigen binding protein is in the form of:
(i) a single domain antibody (sdAb);
(ii) a diabody;
(iii) a triabody;
(iv) a tetrabody;
(v) a Fab;
(vi) a F(ab′)2;
(vii) a Fv;
(viii) a bispecific antibody or other form of multispecific antibody;
(ix) one of (i) to (viii) linked to a constant region of an antibody, Fc or a heavy chain constant domain (CH) 2 and/or CH3; or
(ix) one of (i) to (viii) linked to a protein that binds to an immune effector cell.

15. A fusion protein comprising an antigen binding protein according to any one of claims 1 to 14.

16. A nucleic acid encoding an antigen binding domain according to any one of claims 1 to 14, or a fusion protein according to claim 15.

17. A vector comprising the nucleic acid according to claim 16.

18. A cell comprising a nucleic acid according to claim 16, or a vector according to claim 17.

19. A pharmaceutical composition comprising an antigen binding protein according to any one of claims 1 to 14, a fusion protein according to claim 15, a nucleic acid according to claim 16, a vector according to claim 17, or a cell according to claim 18.

20. A method of reducing pathological thrombus formation, the method comprising administering an antigen binding protein of any one of claims 1 to 14, a fusion protein of claim 15, or a pharmaceutical composition of claim 19, to an individual in need thereof, thereby reducing pathological thrombus formation.

21. A method for inhibiting thrombosis without compromising haemostasis in a subject in need thereof, the method comprising administering an antigen binding protein of any one of claims 1 to 14, a fusion protein of claim 15, or a pharmaceutical composition of claim 19, thereby inhibiting thrombosis without compromising haemostasis in the subject.

22. A method for inhibiting thrombosis in a subject in need thereof comprising administering to the subject an effective dose of an antigen binding protein of any one of claims 1 to 14, a fusion protein of claim 15, or a pharmaceutical composition of claim 19, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.

23. Use of an antigen binding protein of any one of claims 1 to 14, or a fusion protein of claim 15, in the manufacture of a medicament for reducing pathological thrombus formation in a subject in need thereof.

24. Use of an antigen binding protein of any one of claims 1 to 14, or a fusion protein of claim 15, in the manufacture of a medicament for inhibiting thrombosis without compromising haemostasis in a subject in need thereof.

25. Use of an antigen binding protein of ny one of claims 1 to 14, or a fusion protein of claim 15, in the manufacture of a medicament for inhibiting a pathological thrombotic condition in a subject in need thereof.

26. Use of antigen binding protein of any one of claims 1 to 14, or a fusion protein of claim 15, in the manufacture of a medicament for inhibiting thrombosis in a subject in need thereof, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.

27. An antigen binding protein of the invention as described herein for use in reducing pathological thrombus formation in a subject in need thereof.

28. An antigen binding protein of any one of claims 1 to 14, or a fusion protein of claim 15, or a pharmaceutical composition of claim 19, for use in inhibiting thrombosis without compromising haemostasis in a subject in need thereof.

29. An antigen binding protein of any one of claims 1 to 14, or a fusion protein of claim 15, or a pharmaceutical composition of claim 19, for use in inhibiting a pathological thrombotic condition in a subject in need thereof.

30. An antigen binding protein of any one of claims 1 to 14, or a fusion protein of claim 15, or a pharmaceutical composition of claim 19, for use in inhibiting thrombosis in a subject in need thereof, particularly where the thrombosis is associated with: 1) acute coronary syndromes such as myocardial infarction, unstable angina, refractory angina, occlusive coronary thrombus occurring post-thrombolytic therapy or post-coronary angioplasty; 2) ischemic cerebrovascular syndromes including embolic stroke, thrombotic stroke, or transient ischemic attacks; 3) thrombosis occurring in the venous system occurring either spontaneously or in the setting of malignancy, trauma, or surgery, including pulmonary thromboembolism; 4) any coagulopathy including ARDS and DIC, e.g., in the setting of sepsis or other infection, surgery, pregnancy, trauma, or malignancy and whether associated with multi-organ failure or not, thrombotic thrombocytopenic purpura, thromboangiitis obliterans, or thrombotic disease associated with heparin induced thrombocytopenia; 5) thrombotic complications associated with extracorporeal circulation (e.g., renal dialysis, cardiopulmonary bypass or other oxygenation procedure, and plasmaphoresis); 6) thrombotic complications associated with instrumentation (e.g. cardiac or other intravascular catheterization, intraaortic balloon pump, and coronary stent or cardiac valve); and 7) complications associated with fitting of prosthetic devices.


SEQUENCE LISTING (47 sequences)

SEQ ID NO: 1 - protein, 12 aa, Mouse
KSVSTSGYSYMH

SEQ ID NO: 2 - protein, 7 aa, Mouse
LVSNLES

SEQ ID NO: 3 - protein, 9 aa, Mouse
QHIRELTRS

SEQ ID NO: 4 - protein, 5 aa, Mouse
NYWMS

SEQ ID NO: 5 - protein, 17 aa, Mouse
MIHPSDSDTRLNQKFKD

SEQ ID NO: 6 - protein, 7 aa, Mouse
FEGWLPV

SEQ ID NO: 7 - protein, 9 aa, Mouse
GYSFTNYWM

SEQ ID NO: 8 - protein, 6 aa, Mouse
HPSDSD

SEQ ID NO: 9 - protein, 7 aa, Mouse
FEGWLPV

SEQ ID NO: 10 - protein, 107 aa, Mouse
DIELTQSPASLAVSLGQSYRASCKSVSTSGYSYMHWNQQKPGQPPRLLIYLVSNLESGVPARFSGSGSGTDFTLNIHPVEEEDAATYYCQHIRELTRSEGDQAEINR

SEQ ID NO: 11 - protein, 119 aa, Mouse
QVQLQQPGAELVRPGASVKLSCKASGYSFTNYWMSWVKQRPGQGLEWIGMIHPSDSDTRLNQKFKDKATLTVDKSSSTAYMQLSSPTSEDSAGLLLCKKFEGWLPVLLGPRHHSHSLLS

SEQ ID NO: 12 - DNA, 36 bp, Mouse
aagagcgtgtccacctccggctactcttacatgcac

SEQ ID NO: 13 - DNA, 21 bp, Mouse
ctggtgtccaacctggaaagc

SEQ ID NO: 14 - DNA, 27 bp, Mouse
cagcacatcagagagctgaccagaagc

SEQ ID NO: 15 - DNA, 15 bp, Mouse
aactactggatgagc

SEQ ID NO: 16 - DNA, 51 bp, Mouse
atgatccaccccagcgacagcgacacccggctgaaccagaagttcaaggac

SEQ ID NO: 17 - DNA, 21 bp, Mouse
ttcgagggctggctgcccgtg

SEQ ID NO: 18 - DNA, 27 bp, Mouse
ggctacagcttcaccaactactggatg

SEQ ID NO: 19 - DNA, 18 bp, Mouse
caccccagcgacagcgac

SEQ ID NO: 20 - DNA, 21 bp, Mouse
ttcgagggctggctgcccgtg

SEQ ID NO: 21 - DNA, 321 bp, Mouse
gatatcgagctgacacagagccctgccagcctggccgtgtctctgggccagagctatagagccagctgcaagagcgtgtccacctccggctactcttacatgcactggaaccagcagaagcccggccagccccctagactgctgatctacctggtgtccaacctggaaagcggcgtgcccgccagattttctggctctggcagcggcaccgacttcaccctgaacatccaccccgtggaagaagaggacgccgccacctactactgccagcacatcagagagctgaccagaagcgagggcgaccaggccgagatcaataga

SEQ ID NO: 22 - DNA, 357 bp, Mouse
caggtgcagctgcagcagcctggcgctgaactcgtgcggccaggcgcttctgtgaagctgagctgtaaagccagcggctacagcttcaccaactactggatgagctgggtcaagcagaggccaggccagggcctggaatggatcggcatgatccaccccagcgacagcgacacccggctgaaccagaagttcaaggacaaggccaccctgaccgtggacaagagcagcagcaccgcctacatgcagctgtccagccccaccagcgaggattctgctggactgctgctgtgcaagaagttcgagggctggctgcccgtgctgctgggacctagacaccactctcacagcctgctgtct

SEQ ID NO: 23 - protein, 23 aa, Mouse
DIELTQSPASLAVSLGQSYRASC

SEQ ID NO: 24 - protein, 15 aa, Mouse
WNQQKPGQPPRLLIY

SEQ ID NO: 25 - protein, 32 aa, Mouse
GVPARFSGSGSGTDFTLNIHPVEEEDAATYYC

SEQ ID NO: 26 - protein, 9 aa, Mouse
EGDQAEINR

SEQ ID NO: 27 - protein, 30 aa, Mouse
QVQLQQPGAELVRPGASVKLSCKASGYSFT

SEQ ID NO: 28 - protein, 14 aa, Mouse
WVKQRPGQGLEWIG

SEQ ID NO: 29 - protein, 33 aa, Mouse
KATLTVDKSSSTAYMQLSSPTSEDSAGLLLCKK

SEQ ID NO: 30 - protein, 13 aa, Mouse
LLGPRHHSHSLLS

SEQ ID NO: 31 - protein, 25 aa, Mouse
QVQLQQPGAELVRPGASVKLSCKAS

SEQ ID NO: 32 - protein, 17 aa, Mouse
SWVKQRPGQGLEWIGMI

SEQ ID NO: 33 - protein, 42 aa, Mouse
TRLNQKFKDKATLTVDKSSSTAYMQLSSPTSEDSAGLLLCKK

SEQ ID NO: 34 - protein, 13 aa, Mouse
LLGPRHHSHSLLS

SEQ ID NO: 35 - DNA, 69 bp, Mouse
gatatcgagctgacacagagccctgccagcctggccgtgtctctgggccagagctatagagccagctgc

SEQ ID NO: 36 - DNA, 45 bp, Mouse
tggaaccagcagaagcccggccagccccctagactgctgatctac

SEQ ID NO: 37 - DNA, 96 bp, Mouse
ggcgtgcccgccagattttctggctctggcagcggcaccgacttcaccctgaacatccaccccgtggaagaagaggacgccgccacctactactgc

SEQ ID NO: 38 - DNA, 27 bp, Mouse
gagggcgaccaggccgagatcaataga

SEQ ID NO: 39 - DNA, 90 bp, Mouse
caggtgcagctgcagcagcctggcgctgaactcgtgcggccaggcgcttctgtgaagctgagctgtaaagccagcggctacagcttcacc

SEQ ID NO: 40 - DNA, 42 bp, Mouse
tgggtcaagcagaggccaggccagggcctggaatggatcggc

SEQ ID NO: 41 - DNA, 99 bp, Mouse
aaggccaccctgaccgtggacaagagcagcagcaccgcctacatgcagctgtccagccccaccagcgaggattctgctggactgctgctgtgcaagaag

SEQ ID NO: 42 - DNA, 39 bp, Mouse
ctgctgggacctagacaccactctcacagcctgctgtct

SEQ ID NO: 43 - DNA, 75 bp, Mouse
caggtgcagctgcagcagcctggcgctgaactcgtgcggccaggcgcttctgtgaagctgagctgtaaagccagc

SEQ ID NO: 44 - DNA, 51 bp, Mouse
agctgggtcaagcagaggccaggccagggcctggaatggatcggcatgatc

SEQ ID NO: 45 - DNA, 126 bp, Mouse
acccggctgaaccagaagttcaaggacaaggccaccctgaccgtggacaagagcagcagcaccgcctacatgcagctgtccagccccaccagcgaggattctgctggactgctgctgtgcaagaag

SEQ ID NO: 46 - DNA, 39 bp, Mouse; identical to SEQ ID NO: 42

SEQ ID NO: 47 - protein, 208 aa, Human
DISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPT